(19) United States
(12) Patent Application Publication
(10) Pub. No.: US 2026/0364134 A1
(43) Pub. Date: Nov. 19, 2026

(54) AGENT FOR SUPPRESSING AMYLOID BETA AGGREGATION/DEPOSITION AND AGENT FOR SUPPRESSING COGNITIVE FUNCTION DECLINE/IMPROVING COGNITIVE FUNCTION IN A SUBJECT

(71) Applicant: KANEKA CORPORATION, Osaka (JP)

(72) Inventor: Naoki Nishishita, Hyogo (JP)

(73) Assignee: KANEKA CORPORATION, Osaka (JP)

(21) Appl. No.: 19/562,356

(22) Filed: Mar. 10, 2026

Related U.S. Application Data
(63) Continuation of application No. PCT/JP2024/080158, filed on Sep. 11, 2024.

(30) Foreign Application Priority Data
Sep. 11, 2023 (JP) 2023-146708

(51) Int. Cl. A61K 36/61; A61P 25/28; G01N 33/50; G01N 33/68

(52) U.S. Cl. CPC A61K 36/61; A61P 25/28; G01N 33/502; G01N 33/5058; G01N 33/5088; G01N 33/6896; A61K 2236/15; A61K 2236/333

(57) ABSTRACT

An agent for suppressing amyloid β aggregation/deposition and an agent for suppressing cognitive function decline/improving cognitive function in a subject, has therapeutic and/or preventive effects on Alzheimer's disease. The agent for suppressing amyloid β aggregation/deposition or the agent for suppressing cognitive function decline/improving cognitive function in a subject, consists of an extract of a plant belonging to the family Myrtaceae.

### CROSS-REFERENCE TO RELATED APPLICATION

This application claims priority to Japanese Patent Application No. 2023-146708 filed on Sep. 11, 2023, a part or the entire contents of which are incorporate herein by reference.

### TECHNICAL FIELD

One or more embodiments of the present invention relate to an agent for suppressing amyloid β aggregation and/or deposition (hereinafter also referred to as “agent for suppressing amyloid β aggregation/deposition”) and an agent for suppressing cognitive function decline and/or improving cognitive function in a subject (hereinafter also referred to as “agent for suppressing cognitive function decline/improving cognitive function”), which have a high effect of suppressing aggregation/deposition in a method for evaluating amyloid β aggregation suppression and/or deposition suppression (hereinafter also referred to as “aggregation/deposition suppression”) activity on organoids.

### BACKGROUND

Alzheimer's disease (it is often abbreviated to “AD” herein) is a type of irreversible progressive central neurological disease associated with symptoms, such as cognitive impairment (dementia), behavioral disorders, or personality change. While the number of patients with dementia is deduced to be 50,000,000 or greater in 2019 all over the world, approximately 70% of such patients are considered to be patients with AD, and an incidence thereof is increasing. Because of increased medical expenses or problems of nursing care resulting from an increasing number of AD patients, economical or emotional burdens are imposed on the nations or persons involved in patients, and, accordingly, such problem is a serious issue of concern of the society in recent years.

AD is developed as follows. Hydrophobic peptides, amyloid β proteins (often referred to as “Aβ” herein), are aggregated and accumulated in the patient's brain, microtubule-binding proteins, tau proteins, are then hyperphosphorylated to become fibrotic, neurons are destroyed, and the brain is then shrunk (Non-Patent Documents 1 to 3).

On the basis of the mechanism of AD development, a technique of evaluating effects of a test substance added in vitro for inhibiting Aβ aggregation and screening for a candidate compound for treatment of AD has been developed. For example, a method of microliter-scale high throughput screening (which is abbreviated to “MSHTS” herein) of an amyloid β protein aggregation inhibitor using a quantum dot nanoprobe is a cell-free assay-based screening technique that can search for a candidate compound having effects of inhibiting amyloid β protein aggregation in a PBS solvent (Non-Patent Document 4).

As an example of MSHTS, for example, Patent Document 1 discloses a method, an apparatus, and a program for evaluating amyloid formation. Specifically, Patent Document 1 discloses a method for determining inhibitory activity of a test substance on amyloid formation comprising: a step of aggregation reaction comprising allowing an amyloid forming protein, such as an amyloid β protein, to react with a fluorescent probe (e.g., a probe comprising a quantum dot as a fluorescent dye or a quantum dot) capable of binding to an amyloid formed as a result of polymerization of the amyloid forming protein in PBS in the presence or absence of the test substance; a step of imaging comprising obtaining an image of a fluorescence of the aggregation reaction product obtained in the step of aggregation reaction; a step of standard deviation-calculation comprising calculating a standard deviation based on the luminance value of pixels included in an area of interest in the fluorescent image obtained in the step of imaging; and a step of activity evaluation to determine that a test substance has inhibitory activity on amyloid formation when the standard deviation of the luminance in the presence of the test substance is smaller than the standard deviation of the luminance in the absence of the test substance as a result of comparison between the standard deviation of the luminance in the presence of the test substance and the standard deviation of the luminance in the absence of the test substance calculated in the step of calculating a standard deviation.

Patent Document 2 discloses a universal quantum dot nanoprobe used for evaluating amyloid aggregating properties of a protein or peptide and a method for evaluating an amyloid formation inhibitor using such quantum dot nanoprobe. Specifically, Patent Document 2 discloses a quantum dot nanoprobe comprising quantum dots bound to the N- or C-terminus of the amyloid forming peptide via cysteine.

However, inhibitory effects of a candidate compound selected by the cell-free assay system are not observed in the cell-based assay system (the test system using cells) in many occasions. This is considered to occur because the cell-free assay system does not necessarily reflect the in vivo environment. Moreover, even with perilla extract, for example, which has been shown to have an inhibitory effect on Aβ aggregation/deposition in both cell-free and conventional cell-based assays, no consistent effects have been obtained on Aβ aggregation/deposition in the brain; no reliable treatment for Alzheimer's dementia has been established under the current circumstances (Non-Patent Document 5).

Meanwhile, organoids (three-dimensional tissues) are expected to be able to reproduce a physiological environment very similar to that in vivo, including the complex exchange of signals between cells and between cells and the extracellular matrix (ECM) (Non-Patent Document 6).

### PATENT DOCUMENTS

Patent Document 1: WO 2020/138265
Patent Document 2: JP 2017-007990 A

### Non-Patent Documents

Non-Patent Document 1: Hardy J. and Selkoe D. J., 2002, Science, 297 (5580): 353-356
Non-Patent Document 2: Jack C. R. Jr., et al., 2010, Lancet Neurol., 9 (1): 119-128
Non-Patent Document 3: Akira Tamaoka, 2017, Proceedings of the Annual Meeting of the Japanese Research Group on Senile Dementia, Vol. 22, No. 3, pp. 19-23
Non-Patent Document 4: Ishigaki et al., 2013, PLOS ONE, 8 (8): e72992
Non-Patent Document 5: Shimamori et al., 2023, Foods 12 (3): 486
Non-Patent Document 6: Antoni, Burckel, Josset and Noel, Int J Mol Sci., 2015, 16 (3): 5517-5527.

### SUMMARY

Under the above circumstances, an agent for suppressing Aβ aggregation/deposition that can be effective in actual body tissues is provided. More specifically, an agent for suppressing aggregation/deposition for aggregating proteins that exhibit a high aggregation/deposition suppression activity in a screening method for searching for a candidate compound having an effect of suppressing aggregation/deposition of aggregating proteins, such as Aβ, under physiological environments more similar to in vivo using organoids is provided.

An agent for suppressing cognitive function decline/improving cognitive function in a subject is also provided.

The present inventors have conducted concentrated studies and found that it is possible to evaluate whether or not a test substance is a substance having an activity in suppressing or promoting aggregating protein aggregation/deposition by allowing coexistence/culture of aggregating proteins with a label, a test substance, and organoids in a test system using organoids, and quantifying the amount of aggregating proteins aggregated and/or deposited on the surfaces of or inside the organoids using the label as an indicator. This has led to the completion of one or more embodiments of the present invention.

Specifically, one or more embodiments of the present invention include the following.
[1] A method for evaluating a test substance for an activity in suppressing or promoting aggregating protein aggregation/deposition, comprising: a step of allowing coexistence/culture of aggregating proteins with a label, a test substance, and organoids in an aqueous solution; and a step of quantifying the aggregating proteins aggregated and/or deposited on the surfaces of and/or inside the organoids using the label as an indicator.
[2] The method according to [1], wherein the organoids are selected from the group consisting of cerebral organoids, cerebellar organoids, inner ear organoids, thyroid organoids, thymus organoids, T cell mature lymphoid organoids, myocardial organoids, lung organoids, liver organoids, pancreatic organoids, kidney organoids, gastric gland organoids, gut organoids, epithelial organoids, ovarian organoids, testicular organoids, and fused organoids including these organoids.
[3] The method according to [1] or [2], wherein the aggregating proteins are selected from the group consisting of amyloid β protein, tau protein, α-synuclein protein, prion protein, huntingtin protein, amylin protein, apolipoprotein A1, serum amyloid A protein, immunoglobulin light chain, MAP4 protein, β2 microglobulin, TDP-43 protein, and cystatin C protein.
[4] The method according to any one of [1] to [3], wherein the label is an optical label.
[5] The method according to [4], wherein the optical label is a quantum dot.
[6] The method according to any one of [1] to [5], wherein the quantification step includes: a step of photographing an image of the organoids; and a step of calculating a proportion of an area of a fluorescent region from the label, as the aggregating proteins aggregated and/or deposited on the surfaces of and/or inside the organoids, to a surface area of the organoids in the image obtained by the photographing step.
[7] The method according to any one of [1] to [6], wherein the quantification is carried out over time while the organoids are alive.
[8] The method according to any one of [1] to [7], wherein the quantification is carried out for at least 7 days without using genetically modified cells.
[9] An agent for suppressing amyloid β aggregation/deposition, consisting of an extract of a plant belonging to the family Myrtaceae.
[10] The agent for suppressing aggregation/deposition according to [9], which exhibits an effect of suppressing amyloid β aggregation and/or deposition in an evaluation system comprising a step of allowing amyloid β with a label, a test substance, and a cerebral organoid to coexist and culture them and a step of quantifying amyloid β aggregating on a surface of and/or inside the cerebral organoid using the label as an indicator.
[11] The agent for suppressing aggregation/deposition according to [10], which has an suppression rate of 50% or more as compared with a control plot, to which the test substance is not added, in the evaluation system.
[12] The agent for suppressing aggregation/deposition according to any one of [9] to [11], wherein the plant belonging to the family Myrtaceae is guava, Eucalyptus robusta, clove, or allspice.
[13] The agent for suppressing aggregation/deposition according to [12], wherein the extract is an extract obtained by subjecting a pulverized product, which is obtained by pulverizing clove, to solvent extraction.
[14] The agent for suppressing aggregation and/or deposition according to [12] or [13], wherein the extract is an extract obtained by subjecting a pulverized product consisting of particles having a maximum particle size of 5 mm or less, which is obtained by pulverizing clove, to solvent extraction.
[15] The agent for suppressing aggregation and/or deposition according to [12] or [14], wherein the extract is an extract obtained by subjecting a pulverized product consisting of particles having a maximum particle size of 0.5 mm or more, which is obtained by pulverizing clove, to solvent extraction.
[16] The agent for suppressing aggregation and/or deposition according to [13], wherein the extract is an extract obtained by subjecting a pulverized product consisting of particles having a particle size range of 0.5 mm or more and 3 mm or less, which is obtained by pulverizing clove, to solvent extraction.
[17] The agent for suppressing aggregation/deposition according to any one of [9] to [16], which suppresses amyloid β aggregation and/or deposition on a surface of and/or inside a brain tissue cell in a living organism.
[18] An agent for suppressing cognitive function decline/improving cognitive function in a subject, consisting of an extract of a plant belonging to the family Myrtaceae.
[19] The agent for suppressing cognitive function decline/improving cognitive function according to [18], wherein the plant is clove or allspice.
[20] A method for producing an agent for suppressing amyloid β aggregation/deposition, comprising a step of obtaining an extract by subjecting a plant belonging to the family Myrtaceae to solvent extraction.
[21] The method for producing an agent for suppressing aggregation/deposition according to [20], wherein the plant belonging to the family Myrtaceae is guava, Eucalyptus robusta, clove, or allspice.
[22] The method for producing an agent for suppressing aggregation/deposition according to [21], wherein the step of obtaining an extract is a step of obtaining the extract by subjecting a pulverized product, which is obtained by pulverizing clove, to solvent extraction.
[23] The method for producing an agent for suppressing aggregation/deposition according to [22], wherein the pulverized product, which is obtained by pulverizing clove, has a maximum particle size of 5 mm or less.
[24] The method for producing an agent for suppressing aggregation/deposition according to [22] or [23], wherein the pulverized product, which is obtained by pulverizing clove, has a maximum particle size of 0.5 mm or more.
[25] The method for producing an agent for suppressing aggregation/deposition according to [22], wherein the pulverized product, which is obtained by pulverizing clove, has a maximum particle size range of 0.5 mm or more and 3 mm or less.
[26] The method for producing an agent for suppressing aggregation and/or deposition according to any one of [20] to [25], wherein a solvent used in the solvent extraction is an organic solvent, water, or a mixture thereof.
[27] The method for producing an agent for suppressing aggregation/deposition according to any one of [20] to [26], wherein a solvent used in the solvent extraction is an ethanol aqueous solution.
[28] A method for producing an agent for suppressing cognitive function decline/improving cognitive function in a subject, comprising a step of obtaining an extract by subjecting a plant belonging to the family Myrtaceae to solvent extraction.
[29] The production method according to [28], wherein the plant is clove or allspice.
[30] A composition for amyloid β aggregation/deposition suppression, comprising the agent for suppressing aggregation/deposition according to any one of [9] to [17] as an active ingredient.
[31] The composition for aggregation/deposition suppression according to [30], comprising two or more different agents for suppressing aggregation/deposition according to any one of [9] to [17] as active ingredients.
[32] The composition for aggregation/deposition suppression according to [30] or [31], which is used for treating or preventing Alzheimer's dementia or mild cognitive function impairment.

### BRIEF DESCRIPTION OF THE DRAWINGS

FIG. 1 shows photographs indicating the effects of embryoid body (EB) preparation time on organoid-like tissue formation in Example 1.

FIG. 2A shows photographs indicating the state in which cells infiltrate and fill the gel in Example 1.

FIG. 2B is a graph showing the changes in the CCL2 (monocyte chemotactic protein) concentration in the culture supernatant during in-gel culture in Example 1.

FIG. 3 shows photographs indicating the state of Aβ aggregation/deposition in organoids over time in Example 2.

FIG. 4 shows the results of the effect of suppressing Aβ aggregation/deposition of each test specimen evaluated in organoids in Example 3.

FIG. 5 shows the results of the effect of suppressing Aβ aggregation/deposition of each test specimen evaluated in organoids in Example 4.

FIG. 6 shows the results of the effect of suppressing Aβ aggregation/deposition of each test specimen evaluated in organoids in Example 5.

FIG. 7 is a diagram showing the schedule for evaluating the effects of preventing cognitive function decline and improving cognitive function in Example 6.

### DETAILED DESCRIPTION

Hereafter, one or more embodiments of the present invention are described in detail.

### 1. Agent for Suppressing Aβ Aggregation/Deposition

### 1-1. OUTLINE

The first aspect of one or more embodiments of the present invention relates to an agent for suppressing Aβ aggregation/deposition that exhibits amyloid β aggregation/deposition suppression activity. The agent for suppressing Aβ aggregation/deposition of one or more embodiments of the present invention is characterized by exhibiting potent Aβ aggregation/deposition suppression activity in a method for evaluating a test substance for an activity in suppressing or promoting aggregating protein aggregation/deposition, comprising:
a step of allowing coexistence/culture of aggregating proteins with a label, a test substance, and organoids in an aqueous solution; and
a step of quantifying the aggregating proteins aggregated/deposited on the surfaces of and/or inside the organoids using the label as an indicator.

The agent for suppressing Aβ aggregation/deposition of one or more embodiments of the present invention, which was searched for using the evaluation method more similar to an in vivo environment using organoids, can exhibit a favorable effect of suppressing Aβ aggregation/deposition in actual body tissues.

### 1-2. DEFINITIONS OF TERMS

The following terms used herein are defined.

### (1) Organoid

An organoid is a three-dimensional tissue-like structure that exhibits functions similar to those of individual tissues or organs, created through the self-assembly of cells.

Examples of organoids include cerebral organoids, cerebellar organoids, inner ear organoids, thyroid organoids, thymus organoids, T cell mature lymphoid organoids, myocardial organoids, lung organoids, liver organoids, pancreatic organoids, kidney organoids, gastric gland organoids (gastruloids), foregut organoids (organoids that constitute the oral cavity and stomach), gut organoids such as midgut organoids (organoids that constitute the small intestine and ascending colon), hindgut organoids (organoids that constitute parts of the body other than the rectum and ascending colon), epithelial organoids, ovarian organoids, and testicular organoids. Furthermore, a fusion of these organoids may result in a fused organoid that generates new differentiated cells.

### (2) Aggregating Proteins

Aggregating proteins assemble to form an aggregate. The protein that forms the aggregate may be in a liquid or solid state. Types of aggregating proteins are not limited. Examples thereof include polyglutamic acids, disease-associated proteins, and autophagy-associated proteins.

Examples of disease-associated aggregating proteins include causal proteins of Alzheimer's disease, such as the amyloid β protein and the tau protein (including the phosphorylated tau protein), a causal protein of Parkinson's disease, such as the α-synuclein protein, a causal protein of transmissible spongiform encephalopathies (including Creutzfeldt-Jakob disease, bovine spongiform encephalopathies, and prion disease), such as the prion protein, a causal protein of Huntington's disease, such as the huntingtin protein, a causal protein of type II diabetes, such as the amylin protein, a causal protein of arterial sclerosis (including cerebral infarction, pulmonary infarction, and myocardial infarction), such as the apolipoprotein A1 (APOA1 protein), a causal protein of articular rheumatism, such as the serum amyloid A protein, a causal protein of systemic AL amyloidosis, such as the immunoglobulin light chain, a causal protein of myocardial infarction, such as the MAP4 protein, a causal protein of dialysis amyloidosis, such as the β2 microglobulin, and a causal protein of amyotrophic lateral sclerosis, such as the TDP-43 protein. In particular, the amyloid β proteins, such as the amyloid β40 protein, the amyloid β42 protein, the amyloid β43 protein, and the amyloid β38 protein, are preferable.

Aggregating proteins (Main protein misfolding/aggregating diseases) and diseases associated with these aggregating proteins (proteopathies) are shown in Table 1 below.

For example, α-synuclein, TDP-43, prion, cystatin C, amyloid β, and the like are known as aggregating proteins, and are known to be associated with Parkinson's disease, frontotemporal lobar degeneration, prion disease, chronic kidney disease (CKD), Alzheimer's dementia, and diabetic dementia, respectively.

TABLE 1
Association of major aggregating proteins with proteopathies

| | Main protein |
|---|---|
| | misfolding/aggregating |
| Proteopathy | diseases |
| Alzheimer's disease | Amyloid β peptide (Aβ); Tau |
| | protein (see tauopathies) |
| Cerebral β-amyloid angiopathy | Amyloid β peptide (Aβ) |
| Retinal ganglion cell | Amyloid β peptide (Aβ) |
| degeneration in glaucoma | |
| Prion diseases (multiple) | Prion protein |
| Parkinson's disease and other | α-Synuclein |
| synucleinopathies (multiple) | |
| Tauopathies (multiple) | Microtubule-associated protein |
| | tau (Tau protein) |
| Frontotemporal lobar degeneration | TDP-43 |
| (FTLD) (Ubi+, Tau−) | |
| FTLD-FUS | Fused in sarcoma (FUS) protein |
| Amyotrophic lateral | Superoxide dismutase, |
| sclerosis (ALS) | TDP-43, FUS, C9ORF72, |
| | ubiquilin-2 (UBQLN2) |
| Huntington's disease and other | Proteins with tandem glutamine |
| trinucleotide repeat disorders | expansions |
| (multiple) | |
| Familial British dementia | ABri |
| Familial Danish dementia | ADan |
| Hereditary cerebral | Cystatin C |
| hemorrhage with amyloidosis | |
| (Icelandic) (HCHWA-I) | |
| CADASIL | Notch3 |
| Alexander disease | Glial fibrillary acidic |
| | protein (GFAP) |
| Pelizaeus-Merzbacher disease | proteolipid protein (PLP) |
| Seipinopathies | Seipin |
| Familial amyloidotic neuropathy, | Transthyretin |
| Senile systemic amyloidosis | |
| Serpinopathies (multiple) | Serpins |
| AL (light chain) amyloidosis | Monoclonal immunoglobulin |
| (primary systemic amyloidosis) | light chains |
| AH (heavy chain) amyloidosis | Immunoglobulin heavy chains |
| AA (secondary) amyloidosis | Amyloid A protein |
| Type II diabetes | Islet amyloid polypeptide |
| | (IAPP; amylin) |
| Aortic medial amyloidosis | Medin (lactadherin) |
| ApoAI amyloidosis | Apolipoprotein AI |
| ApoAII amyloidosis | Apolipoprotein AII |
| ApoAIV amyloidosis | Apolipoprotein AIV |
| Familial amyloidosis of the Finnish | Gelsolin |
| type (FAF) | |
| Lysozyme amyloidosis | Lysozyme |
| Fibrinogen amyloidosis | Fibrinogen |
| Dialysis amyloidosis | Beta-2 microglobulin |
| Inclusion body myositis/myopathy | Amyloid β peptide (Aβ) |
| Cataracts | Crystallins |
| Retinitis pigmentosa with rhodopsin | rhodopsin |
| mutations | |
| Medullary thyroid carcinoma | Calcitonin |
| Cardiac atrial amyloidosis | Atrial natriuretic factor |
| Pituitary prolactinoma | Prolactin |
| Hereditary lattice corneal dystrophy | Keratoepithelin |
| Cutaneous lichen amyloidosis | Keratins |
| Mallory bodies | Keratin intermediate filament |
| | proteins |
| Corneal lactoferrin amyloidosis | Lactoferrin |
| Pulmonary alveolar proteinosis | Surfactant protein C (SP•C) |
| Odontogenic (Pindborg) tumor | Odontogenic ameloblast-associated |
| amyloid | protein |
| Seminal vesicle amyloid | Semenogelin I |
| Apolipoprotein C2 amyloidosis | Apolipoprotein C2 (ApoC2) |
| Apolipoprotein C3 amyloidosis | Apolipoprotein C3 (ApoC3) |
| Lect2 amyloidosis | Leukocyte chemotactic factor-2 |
| | (Lect2) |
| Insulin amyloidosis | Insulin |
| Galectin-7 amyloidosis (primary | Galectin-7 (Gal7) |
| localized cutaneous amyloidosis) | |
| Corneodesmosin amyloidosis | Corneodesmosin |
| Enfuvirtide amyloidosis | Enfuvirtide |
| Cystic fibrosis | cystic fibrosis transmembrane |
| | conductance regulator (CFTR) |
| | protein |
| Sickle cell disease | Hemoglobin |
| Myocardial Infarction | MAP4 |

Examples of autophagy-associated proteins include the ubiquitin-like proteins, such as Atg-8 and Atg-12.

The aggregating proteins may be selected from the group consisting of naturally-occurring proteins existing in nature, modified proteins derived from naturally-occurring proteins by artificial variation or modification, or artificial proteins comprising artificially designed amino acid sequences.

### (3) Aggregation/Aggregate

As used herein, the term “aggregation” refers to a state in which peptide or protein molecules (which may be modified with sugar chains or lipids) are aggregated together, and may be water-soluble or water-insoluble.

The term “aggregate” used herein refers to an assembly of two or more aggregating proteins. A so-called “protein complex” is within the scope of the “aggregate” herein.

An aggregate may be a homo-aggregate consisting of a single type of proteins or a hetero-aggregate consisting of different types of proteins.

### (4) Deposition

As used herein, “deposition” of aggregating proteins on the surfaces of and/or inside organoids refers to the immobilization of aggregating proteins to the surfaces of and/or inside organoids. The deposition site may be on the surfaces of and/or inside cells that form the organoid, or it may be deposited between the cells and the ECM.

### (5) Cell

In one or more embodiments of the present invention, cells that form organoids or cells from which organoids originate are described below.

A cell may be derived from a multicellular organism. An animal-derived cell is preferable, and a mammalian-derived cell is more preferable. Examples of animals include rodents, such as mice, rats, hamsters, and guinea pigs, livestock or pet animals, such as dogs, cats, rabbits, cows, horses, sheep, and goats, and primates, such as humans, rhesus monkeys, gorillas, and chimpanzees. A human-derived cell is particularly preferable.

A cell type is not limited. Examples thereof include a body-tissue-derived cell, a cell derived from a body-tissue-derived cell, a stem cell, a cell differentiated from a stem cell.

The term “body tissue” refers to various tissue constituting the body of an organism. Examples thereof include epithelial tissue, connective tissue, muscle tissue, and nerve tissue.

The term “stem cell” refers to a cell having the potential to differentiate into different types of cells and the potential to self-renew. Examples thereof include an adult stem cell and a pluripotent stem cell.

The “adult stem cell” is a stem cell that exists in various tissue in an adult, the terminal differentiation thereof is incomplete, and such stem cell has a certain degree of pluripotency. An adult stem cell is also referred to as a somatic stem cell or tissue stem cell. Examples thereof include a mesenchymal stem cell, a neural stem cell, an intestinal epithelial stem cell, a hematopoietic stem cell, a hair follicle stem cell, a pigment stem cell, and a cancer stem cell.

The term “pluripotent stem cell” refers to a cell having a pluripotency to differentiate into all types of cells constituting an organism and capable of indefinitely growing while maintaining the pluripotency in in vitro culture under adequate conditions. Examples thereof include an embryonic stem (ES) cell, an embryonic germ stem cell, a germ stem cell, and an induced pluripotent stem (iPS) cell.

“Differentiated cells” refer to cells that constitute a living organism and can be cultured in vitro under appropriate conditions. Examples thereof include salivary gland mucous cells, salivary gland nl cells, von Ebner's gland cells in taste buds, mammary gland cells, lacrimal gland cells, auditory canal gland cells, eccrine gland cells, apocrine gland cells, eyelash gland cells, sebaceous gland cells, Bowman's gland cells, Brunner's gland cells, seminal vesicle cells, bulbourethral gland cells, goblet cells, endometrial cells, gastric mucosal cells, parietal cells, pancreatic acinar cells, type II pneumocytes in the lung, hormone-secreting cells, anterior pituitary cells, growth hormone-secreting cells, prolactin-producing cells, pituitary thyrotrophs, adrenocorticotropic cells, melanocyte-stimulating hormone-producing cells, large neurosecretory neurons, astrocytes, microglia, cardiomyocytes, atrial myocytes, oxytocin-secreting cells, vasopressin-secreting cells, serotonin-secreting cells, endorphin-secreting cells, insulin-secreting cells, thyroid cells, follicular cells, parafollicular cells, parathyroid cells, eosinophilic cells, granulosa lutein cells, corneal epithelial cells, bulbourethral gland cells, pyramidal cells, autonomic neurons, cholinergic neurons, adrenergic neurons, peptidergic neurons, renal juxtamyocellular cells, glomerular epithelial cells, hepatocytes, adipocytes, white adipocytes, brown adipocytes, ameloblasts, corneal fibroblasts, tendon fibroblasts, smooth muscle cells, neutrophils, eosinophils, basophils, regulatory T cells, cytotoxic T cells, natural killer T cells, B cells, natural killer cells, reticulocytes, erythroblasts, hepatocytes, T cells, neurons, and hormone-secreting cells.

In addition, neurons induced to differentiate from iPS cells derived from Alzheimer's disease (AD) patients can also be particularly suitably used to form cerebral organoids.

### (6) Extracellular Matrix: ECM

As used herein, the term “extracellular matrix” refers to a matrix that embeds cells. The extracellular matrix may be any gel-like substrate capable of embedding cells, and may be a mixed protein of animal origins, such as Matrigel, or a polymer-derived substrate such as collagen, fibronectin, entactin, laminin, vitronectin, or an artificial protein.

### (7) Medium

In one or more embodiments of the present invention, a “medium” is a liquid or solid substance prepared to culture cells. In principle, a medium comprises the minimal amount or more ingredients essential for growth and/or maintenance of cells. A medium may be a basal medium or a special cell culture medium.

A “basal medium” is a medium that constitutes a base of a medium for various animal cells. Culture can be performed in a basal medium, or various culture additives can be added to a basal medium to prepare a medium specific to any of various cells in accordance with purposes (i.e., a special cell culture medium). Examples of basal media that can be used include, but are not particularly limited to, Neurobasal medium, BME medium, BGJb medium, CMRL1066 medium, Glasgow MEM medium, Improved MEM Zinc Option medium, Iscove's Modified Dulbecco's (IMDM) medium, Medium 199 medium, Eagle MEM medium, αMEM medium, Dulbecco's Modified Eagle's (DMEM) medium, Ham's F10 medium, Ham's F12 medium, RPMI 1640 medium, Fischer's medium, and a medium mixture of any thereof (e.g., DMEM/F12 medium). In addition, media that are used for human iPS cell or human ES cell culture may be used.

A “special cell culture medium” is a medium optimized for culture of special cells with the addition of various supplements to the basal medium or a medium prepared to be capable of inducing cells to differentiate into particular cells. Examples include nerve cell culture media commercialized by various manufacturers. A specific example is “M medium,” which is obtained by adding, as supplements, NGF2.5S and BDNF to a nerve cell culture medium (Sumitomo Bakelite Co., Ltd.) that has been prepared by adding a culture supernatant of primary astroglia cells cultured on a nutritive medium and serum albumin to a basal medium prepared by adding insulin and transferrin to DMEM/F12 (5:5). A culture medium for pluripotent stem cells, such as human iPS cells or human ES cells, is also within the scope of the special cell culture medium.

A medium may or may not contain serum (i.e., a serum-free medium).

### (8) Label

The term “labeling” used herein refers to modification of a target substance to identify the target substance. The target substance can be detected or selected easily with certainty by labeling.

Labeling is performed in accordance with a type of a target substance. Since aggregating proteins are to be detected herein, the type of the target substance is a protein. Accordingly, any means of labeling that can directly or indirectly label a protein is employed. Examples of direct labeling include a method of labeling comprising binding a label substance to aggregating proteins and a method of labeling comprising expressing aggregating proteins as a fusion protein thereof with a label peptide. An example of indirect labeling is a method of labeling an antibody that specifically recognizes and binds to aggregating proteins or an active fragment thereof directly or indirectly via a secondary antibody. A method for binding a label substance is preferable.

A label substance used for protein labeling is not limited. Examples thereof include an optical label.

The term “optical label” used herein refers to a label with a fluorescent, luminescent, or other substance that emits light, such as visible light, near infrared light, or near-ultraviolet light.

A “fluorescent substance” is a substance that forms an excited state upon absorption of an excited light at a particular wavelength and emits fluorescence when it returns to the ground state. Fluorescent substances encompass a fluorescent dye and a fluorescent protein.

Examples of fluorescent dyes include quantum dot, FITC, Texas, Texas Red, Alexa Fluor 405, Alexa Fluor 488, Alexa Fluor 647, Alexa Fluor 700, Pacific Blue, DyLight 405, DyLight 550, DyLight 650, phycoerythrin-cyanin 5 (PE-Cy5), phycoerythrin-cyanin 7 (PE-Cy7), phycoerythrin (PE), peridinin chlorophyll protein (PerCP), peridinin chlorophyll protein-cyanin 5.5 (PerCP-Cy5.5), cy3, cy5, cy7, FAM, HEX, VIC (registered trademark), JOE, ROX, TET, Bodipy493, NBD, TAMRA, Quasar (registered trademark) 670, Quasar (registered trademark) 705, Allophycocyanin (APC), congo red, thioflavin T, thioflavin S, Fluorescamine or a derivative thereof, fluorescein or a derivative thereof, azo compound, rhodamine or a derivative thereof, coumarin or a derivative thereof, pyrene or a derivative thereof, and cyanine or a derivative thereof, with a quantum dot being preferable.

A “quantum dot” (which is often referred to as “QD” herein) is a nanoscale semiconductor crystal having quantum mechanics-based optical properties and emitting visible light and fluoresce in a near-infrared region. In general, a quantum dot is 2 nm to 10 nm in diameter and is composed of approximately 10 to 50 atoms. A quantum dot is excellent in properties, such that a large number of fluorescent colors are obtained depending on particle diameters, and fluorescence fading is less likely to occur. Accordingly, application thereof is advancing as a biosensing material and a cell or animal imaging material. Examples of quantum dots include semiconductor quantum dots, in particular, core-shell CdSe/ZnS quantum dots, such as Qdot (registered trademark) 525, Qdot 545, Qdot 565, Qdot 585, Qdot 605, Qdot 655, Qdot 705, and Qdot 800 (Thermo Fisher Scientific).

An example of a “fluorescent protein” is GFP.

### (9) Test Substance

Examples of test substances include nucleic acids, RNAs, peptides, proteins, antibodies, synthetic compounds, cells, cell extracts, cell culture supernatants, exosomes, microbial metabolites, plant extracts, and seaweed extracts.

### (10) Treatment and Prevention

As used herein, the term “treatment” refers to curing a disease, suppressing or preventing the progression or chronicity of disease symptoms, and alleviating, ameliorating, or remitting disease symptoms.

In addition, the term “prevention” as used herein refers to suppressing or inhibiting the onset of a particular disease.

### (11) Plant Extract

As used herein, the term “plant extract” refers to a mixture of a plurality of kinds of components obtained from a plant. Typically, plant extracts correspond to plant components such as low-molecular-weight compounds and/or soluble components extracted from a plant body into a solvent, or plant components including proteins, nucleic acids, lipids, sugars, and low-molecular-weight compounds contained in the squeezed juice of a plant body. All or part of the components contained in a plant extract may be unidentified components. The plant extract may be in any state. The plant extract may be in a liquid state, a solid state (including a powder state or a granular state), or a paste state.

As used herein, the term “squeezed juice” refers to a liquid component obtained by squeezing a plant body. The squeezed juice may contain small fragments of a plant body as a solid component, or may consist of a liquid component of a plant body after filtration.

### (12) Plant Body

As used herein, the term “plant body” refers to the entire part (whole plant) or part of a plant that constitutes an individual plant. The part of the plant is not particularly limited. Examples thereof include leaves, stems, buds, leaf sheaths, petioles, aerial tubers, rhizomes (including corms, scaly bulbs/bulbs, rootstocks, and tubers), roots (including tuberous roots and aerial roots), seeds, hypocotyls, fruits, and flower buds. The plant body may be in a fresh, fermented, or dried state. The state of the plant body may be appropriately selected depending on the morphology of the plant body. For example, when the plant body is in a powder state, it may be in a dried state. In the case of squeezed juice, it may be in a fresh or fermented state.

### 1-3. ORGANOID PRODUCTION

An example of organoid production is shown below.

Examples of the organoid production method include a method comprising a step of culturing cells to form a spheroid, a step of embedding the spheroid obtained in the spheroid formation step in a gel, and a step of culturing the spheroid embedded in the gel in the gel embedding step. According to such a method, it is possible to obtain an organoid that can be used in selecting (screening) a candidate substance that exhibits activity in suppressing or promoting aggregating protein aggregation/deposition.

The steps in the method for producing these organoids are described below.

### 1-3-1. Spheroid Formation Step

The “spheroid formation step” is a step of culturing cells to form a spheroid such as an embryoid body.

The shape of a culture vessel used in this step is not particularly limited, but examples thereof include culture vessels in dish, flask, well, bag, and spinner flask shapes.

The seeding density of cells in a culture medium in the culture vessel (cell density at the start of culture) can be adjusted as appropriate, but may be, for example, 0.01×10⁵ cells/ml or more, 0.1×10⁵ cells/ml or more, or 1× 10⁵ cells/ml or more, and 20×10⁵ cells/ml or less, or 10×10⁵ cells/ml or less.

The temperature conditions for the culture are, for example, 25 °C to 40 °C, 30 °C to 39 °C, or 34 °C to 38 °C.

Meanwhile, the culture time may be, for example, 10 hours or more, 12 hours or more, or 20 hours or more, and 300 hours or less, 200 hours or less, or 100 hours or less. When the culture time is excessively long, exceeding 100 hours, the bonds between cells may become excessively strong, and the cells may not be able to fill the gel well during the subsequent in-gel culture step of the spheroid embedded in the gel.

The culture may be static culture or culture under conditions where the medium is flowing (flow culture), but static culture is preferred.

In this step, for example, embryoid bodies may be formed during the process of inducing iPS cells to become neurons, or embryoid bodies may be formed from iPS cells, and then embryoid bodies of neurons may be formed.

When forming embryoid bodies during the process of inducing iPS cells into neurons, iPS cells are first cultured in a neural progenitor cell induction medium (e.g., medium prepared by adding STEMdiff (trademark) SMADi Neural Induction Supplement to STEMdiff (trademark) Neural Induction Medium (hereinafter referred to as “NIMS medium”)) and induced to differentiate into neural progenitor cells. In this case, a ROCK inhibitor may be added to the medium.

The ROCK inhibitor is defined as a substance that inhibits the kinase activity of Rho-kinase (ROCK: Rho-associated protein kinase). Examples thereof include Y-27632 (4-[(1R)-1-aminoethyl]-N-pyridin-4-ylcyclohexane-1-carboxamide or a salt thereof (e.g., dihydrochloride salt)), H-1152 ((S)-(+)-2-methyl-1-[(4-methyl-5-isoquinolinyl) sulfonyl]-hexahydro-1H-1,4-diazepine or a salt thereof (e.g., dihydrochloride salt)), Fasudil/HA1077 (1-(5-isoquinolinesulfonyl) homopiperazine or a salt thereof (e.g., dihydrochloride salt)), Wf-536 ((+)-(R)-4-(1-aminoethyl)-N-(4-pyridyl)benzamide monohydrochloride), Y39983 (4-[(1R)-1-Aminoethyl]-N-1H-pyrrolo[2,3-b]pyridin-4-ylbenzamide dihydrochloride), SLx-2119 (2-[3-[4-(1H-indazol-5-ylamino)-2-quinazolinyl]phenoxy]-N-(1-methylethyl)-acetamide), Azabenzimidazole-aminofurazans, DE-104, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, and VAS-012.

The final concentration of the ROCK inhibitor in the medium may be, for example, 5 μM or more, or 20 μM or more, and 100 μM or less, or 50 μM or less.

The resulting neural progenitor cells are then expanded in a neural progenitor cell culture medium (e.g., STEMdiff (trademark) Neural Progenitor Medium). The neural progenitor cells are cultured in a neuron culture medium (e.g., a mixed medium of Neurobasal Medium, minus phenol red (Thermo Fisher Scientific) with B-27 supplement (Thermo Fisher Scientific) and Penicillin-Streptomycin Mixed Solution (Nacalai Tesque) (hereinafter referred to as “NbM medium”)) for the specified culture time described above, and are induced to differentiate into neurons while forming embryoid bodies. The medium is further supplemented with a hedgehog protein (e.g., human recombinant sonic hedgehog (SHH, manufactured by VERITAS Corporation)) as an essential component. The final concentration of the hedgehog protein in the medium may be, for example, 1 ng/ml or more, 10 ng/ml or more, and 200 ng/ml or less, or 100 ng/ml or less.

Meanwhile, when forming embryoid bodies from iPS cells and then forming embryoid bodies of neurons, the iPS cells are cultured in an embryoid body formation medium (e.g., EB Formation medium in the STEMdiff (trademark) Cerebral Organoid Kit) for the above-described specified culture time to form embryoid bodies from the iPS cells. The embryoid bodies of iPS cells are then cultured in a neuronal differentiation induction medium (e.g., GMEM (Thermo Fisher Scientific) containing 5% KSR (Thermo Fisher Scientific) and AGN193109 (Sigma-Aldrich Japan)) to induce differentiation into neurons, and embryoid bodies of neurons can be obtained.

Whether or not neurons have been induced to differentiate can be determined using neural progenitor cell markers and/or neuron markers as indicators. An example of a neural progenitor cell marker is nestin. Furthermore, an example of a neuron marker is MAP2.

The neural progenitor cell marker and/or the neuron marker can be detected by any detection method known in the art. Examples of a method for detecting an expression marker include, but are not limited to, flow cytometry. In flow cytometry using a fluorescence-labeled antibody, when cells that emit stronger fluorescence compared to a negative control (isotype control) are detected, the cells are determined to be “positive” for the marker. The ratio of cells that are positive for a fluorescence-labeled antibody analyzed by flow cytometry is sometimes referred to as the positive rate. Any antibody known in the art can be used as the fluorescence-labeled antibody. Examples thereof include, but not limited to, antibodies labeled with fluorescein isothiocyanate (FITC), phycoerythrin (PE), allophycocyanin (APC), and the like.

Neural progenitor cell markers and/or neuron markers can be detected by immunostaining reactions. For example, cells or embryoid bodies in a culture vessel are reacted with a primary antibody, then with a fluorescence-labeled secondary antibody, and cells or embryoid bodies that visually emit fluorescence are detected by observation under a fluorescence microscope. For example, the ratio of cells or embryoid bodies stained with a fluorescence-labeled secondary antibody to cells or embryoid bodies stained with DAPI (a cell nuclear marker) can be taken as the positive rate. Any antibody known in the art can be used as the fluorescence-labeled secondary antibody, and examples thereof include, but are not limited to, antibodies labeled with Alexa Fluor 488 and the like.

For example, when the proportion (ratio) of cells or embryoid bodies that are positive for neural progenitor cell markers may be, for example, 10% or less, or 5% or less, and/or the proportion (ratio) of cells or embryoid bodies that are positive for neuron markers may be 70% or more, or 80% or more, it can be determined that neurons have been significantly induced to differentiate.

### 1-3-2. Gel Embedding Step

In this step, the spheroids obtained in the spheroid formation step are embedded in a gel. For example, the spheroids are embedded in a gel such as Matrigel. An example of the embedding method is to attach parafilm to a culture plate, create a depression using the chip attachment part or the like, place a spheroid in the depression, drop gel therein, leave the gel to stand for, for example, 10 to 720 minutes (or 30 to 180 minutes), and then collect the gel embedding the spheroid into a new culture plate.

### 1-3-3. In-Gel Culture Step

In this step, the spheroid embedded in the gel embedding step is cultured in the gel.

The gel-embedded spheroid is subjected to shaking culture in a culture vessel containing a medium. For example, shaking culture is performed by rotation in a bioshaker at 10 to 200 rpm (or 30 to 150 rpm).

The temperature conditions for the culture are, for example, 25 °C to 40 °C, 30 °C to 39 °C, or 34 °C to 38 °C.

Meanwhile, the culture time may be, for example, 14 days or more, or 21 days or more, and 365 days or less, or 280 days or less, such that the cells sufficiently infiltrate and fill the gel, thereby forming organoids.

In addition, the maturity of organoids can be determined by quantifying cell migration proteins contained in the culture supernatant during the culture period, during which cells infiltrate and fill the gel from the spheroids embedded in the gel.

Therefore, the in-gel culturing step may include measuring the concentration of cell migration proteins in the culture supernatant.

Examples of cell migration proteins include CCL2 (monocyte chemotactic protein).

The concentration of cell migration proteins in the culture supernatant can be measured by an immunological measurement method such as ELISA.

For example, when the concentration of CCL2 in the culture supernatant may be 500 μg/ml or less, or 200 μg/ml or less, it can be determined that the filling of the gel with cells is complete and organoids have been formed.

### 1-4. METHOD FOR EVALUATING TEST SUBSTANCE FOR ACTIVITY IN SUPPRESSING OR PROMOTING AGGREGATING PROTEIN AGGREGATION/DEPOSITION

Each step of the method of this aspect are described below.

### 1-4-1. Step of Coexistence and Culture (Hereinafter Also Referred to as “Coexistence/Culture Step”)

The “coexistence/culture step” is a step of allowing aggregating proteins with a label, a test substance, and organoids to coexist in an aqueous solution and culturing (incubating) the organoids in the coexistence thereof. This step allows aggregating proteins labeled through culture (incubation) to be aggregated and/or deposited on the surfaces of and/or inside the organoids. The term “culture (incubation)” as used herein does not necessarily mean increasing the number of cells, but simply means maintaining the cells in the coexistence for a certain period of time or longer. In a separate control experiment, organoids may coexist with labeled aggregating proteins in an aqueous solution (without a test substance), and the organoids may be cultured (incubated) in the coexistence thereof.

Examples of the aqueous solution used herein include culture media, physiological saline, and Ringer's solution.

For example, in a culture vessel (e.g., a plate such as a 96-well microplate), a solution containing a labeled aggregating proteins diluted with a medium or the like and a solution containing a test substance diluted with a medium or the like are added to the organoids. A solution containing unlabeled aggregating proteins diluted with a medium or the like may be further added. The addition of these solutions can be done simultaneously or sequentially. The order of addition may be in any order.

Alternatively, organoids are cultured (incubated) in the presence of labeled aggregating proteins, which causes the labeled aggregating proteins to be aggregated and/or deposited on the surfaces of and/or inside the organoids. In this way, the addition of labeled aggregating proteins aggregated and/or deposited in organoids and a solution containing a test substance allows the coexistence/culture of the labeled aggregating proteins, the test substance, and the organoids.

In a culture vessel, for example, for organoids having a diameter of 1 mm or more, the respective concentrations of the aggregating proteins and the test substance in the aqueous solution (culture medium) may be, for example, as follows: labeled aggregating protein: final concentration of 5 to 100 nM (or 10 to 50 nM); test substance: final concentration of 1 to 100 ng/μl (or 10 to 50 ng/μl); and unlabeled aggregating protein: final concentration of 5 to 100 μM (or 10 to 50 μM).

The conditions for coexistence and culture (incubation) are not particularly limited as long as the labeled aggregating proteins can be aggregated and/or deposited on the surfaces of and/or inside the organoids. The temperature is, for example, 25 °C to 40 °C, 30 °C to 39 °C, or 34 °C to 38 °C. The time period may be, for example, 30 minutes to 1200 hours (50 days), 12 hours to 96 hours (4 days), 24 hours (1 day) to 72 hours (3 days), or 36 hours to 48 hours (2 days).

The culture (incubation) may be static culture or culture under conditions where the medium is flowing (flow culture), but static culture is preferred.

### 1-4-2. Quantification Step

The “quantification step” is a step of quantifying the aggregating proteins aggregated and/or deposited on the surfaces of and/or inside the organoids obtained in the coexistence/culture step using, as an indicator, the label attached to the added aggregating proteins after the culture (incubation) or over time (e.g., every 1 to 50 days, or every 3 to 40 days) while the organoids are alive (while the culture is continued). In this step, the aggregation/deposition of aggregating proteins on the surfaces of and/or inside the organoids is quantitatively detected.

For example, in a case in which the aggregating proteins are labeled with an optical labeling substance such as quantum dots, aggregation/deposition can be detected by fluorescence observation using a fluorescence microscope. When fluorescence is observed on the surfaces of and/or inside the organoids, it can be determined that aggregates/deposits have formed. The fluorescence intensity is then quantified, and the presence or absence of aggregates/deposits is quantitatively detected, for example, by comparing the fluorescence intensity value with that of a negative control.

Specifically, the quantification step may include: a step of photographing an image of organoids; and a step of calculating a proportion of an area of a fluorescent region from a label, such as quantum dots, as aggregating proteins aggregated and/or deposited on the surfaces of and/or inside the organoids, to a surface area of the organoids in the image obtained by the photographing step using the fluorescence from the label as an indicator.

For example, in a case in which quantum-dot-modified amyloid β proteins (e.g., amyloid β₄₀ protein) and unlabeled amyloid β proteins (e.g., amyloid β₄₂ protein) are added to the cerebral organoids, the cerebral organoids are irradiated with light and the quantum dots are used to visualize aggregates/deposits containing the unlabeled amyloid β proteins and the quantum-dot-modified amyloid β proteins on the surfaces of and/or inside the cerebral organoids.

Next, an image of the cerebral organoids is acquired, and the area of the fluorescent region is calculated from image analysis of the fluorescence from the quantum dots. The area of the fluorescent region positively correlates with the amount of aggregated/deposited aggregating proteins. Therefore, for example, it is evaluated whether or not a test substance added has aggregation/deposition suppression activity or aggregation/deposition promotion activity on aggregating proteins from a comparison of the proportion of the area of the fluorescent region to the cerebral organoid surface area with a negative control (e.g., in the absence of the test substance) and/or a positive control (e.g., in the presence of a known substance with aggregation/deposition suppression activity or with aggregation/deposition promotion activity).

For example, an image of the organoids is photographed, and the organoid area in the image obtained by photographing is calculated (organoid surface area).

The area of the fluorescent region of the aggregating proteins in the image is then calculated (the deposition area of the aggregating proteins).

The aggregation/deposition rate of aggregating proteins in organoids can be calculated using the following Formula (1):

Aggregation/deposition rate of aggregating proteins in organoids(%)=(Aggregation/deposition area of aggregating proteins/organoid surface area)×100 (1).

Furthermore, when this test system is established, by setting up a group to which the test specimen is also added simultaneously, it is possible to quantitatively analyze the effect of the specimen on the suppression of aggregation/deposition of aggregative proteins in organoids. For example, the aggregating protein aggregation/deposition suppression function of the test substance can be quantified as the aggregation/deposition suppression rate (%), which can be calculated using the following Formula (2):

Aggregating protein(Aβ in this example)aggregation/deposition suppression rate (%)=(Aggregating protein aggregation/deposition rate in the absence of test substance(%))−(Aggregating protein aggregation/deposition rate in the presence of test substance(%)) (2).

It can be seen that the larger the aggregation/deposition suppression rate (%) calculated by Formula (2), the greater the aggregation/deposition suppression effect of the test substance.

Thus, in a case in which the proportion of the area of the fluorescent region to the organoid surface area is lower than that for a negative control, the added test substance can be determined to have aggregation/deposition suppression activity. In a case in which the proportion of the area of the fluorescent region to the organoid surface area is not more than that for a positive control (e.g., in the presence of a known substance with aggregation/deposition suppression activity), the added test substance can be determined to have aggregation/deposition suppression activity.

### 1-4-3. Effects

A substance evaluated to have aggregation/deposition suppression activity by the method of this aspect is thought to have activity in a state more similar to an in vivo environment. Therefore, the substance is expected to be useful as a substance/compound (drug, supplement, functional food, health food, general food, or the like) for the treatment and prevention of various diseases caused by aggregating proteins, including amyloidosis such as AD and Parkinson's disease.

### 1-5. AGENT FOR SUPPRESSING Aβ AGGREGATION/DEPOSITION

### 1-5-1. Constitution

### (1) Plant Extract

The agent for suppressing Aβ aggregation/deposition of one or more embodiments of the present invention comprises a plant extract. The plant extract of one or more embodiments of the present invention may be obtained by any extraction method or in any state, as long as it is an extract obtained from a plant body. For example, examples thereof include: liquid components (so-called extract liquid) obtained by immersing a plant body in a solvent such as water, an organic solvent (such as ethanol), or a mixture thereof at a predetermined temperature for a predetermined time, and then removing the plant body by centrifugation or filtration; solids (including granules and powders) remaining after the solvent is removed from the extract liquid by reducing pressure, evaporation, drying, or the like; and pastes in an intermediate state thereof. Other examples include squeezed juice obtained by squeezing a plant body, a resulting product obtained by filtering the squeezed juice to remove a solid component, and a solid obtained by evaporating/drying or the like the water content thereof.

The extraction method using a solvent may be a known extraction method. Examples of the solvent used for extraction include water, an organic solvent, and a mixture of water and a water-soluble organic solvent such as ethanol. When a mixed solution of ethanol and water (ethanol aqueous solution) is used, the ethanol concentration of the mixed solution can be appropriately selected. For example, the ethanol concentration may be 65% (w/w %) or more, 75% (w/w %) or more, or 85% (w/w %) or more, and it is particularly preferable to select an ethanol concentration in a range of 85% to 99% (w/w %). When the solvent is water, the solvent temperature during extraction may be in a range of 10 °C to 100 °C, 40 °C to 99 °C, or 70 °C to 99 °C under 1 atmosphere, or in a range of 100 °C to 121 °C under 1 to 2 atmospheres using a heat-resistant and pressure-resistant sealed vessel such as an autoclave. When the solvent is ethanol, the temperature may be in a range of 0 °C to 78.3 °C, 15 °C to 77 °C, or 20 °C to 77 °C under 1 atmosphere. Furthermore, when the solvent is a mixture of water and ethanol, the conditions for water or ethanol described above may be applied appropriately depending on the concentration of ethanol, for example, 90% (w/w %). The extraction time varies depending on the temperature of the solvent. In general, when the solvent temperature is a low temperature below 40 °C, a long time, for example, 1 to 10 days or more, is required, whereas when the solvent temperature is a high temperature at 40 °C or higher and below the boiling point under 1 atmosphere, a short time, for example, 2 minutes to 72 hours is sufficient.

When preparing a plant extract by extraction, juice-squeezing, or the like, the plant body used may be in its intact state, or may be in a fragmented or powderized state. The “plant in its intact state” refers to the entire plant when the whole plant is used, or to the state of some of the organs when a part of the plant, such as the flower, leaf, or root, is used. Meanwhile, the “fragmented” state refers to a plant body cut into small pieces by cutting, grinding, or the like. The size of the fragments is not particularly limited, but a small size is preferable to improve the efficiency of extraction into the solvent and the efficiency of juice-squeezing. In addition, an appropriate pulverization size (range of maximum particle size) can be selected, taking into consideration the operability during production and the Aβ aggregation/deposition suppression rate. For example, the range may be 0.15 to 5 mm, or 0.2 to 3 mm. The “powderized” state refers to a plant body powderized as is or after being frozen, dried, or lyophilized, and then pulverized. The size of the particles constituting the powder is not particularly limited, and may be, for example, a granular size, a fine powder size, or a size including both thereof. Specifically, the maximum particle size may be 6 mm or less, 5 mm or less, 4 mm or less, 3 mm or less, and the lower limit of the maximum particle size may be 0.1 mm or more, 0.2 mm or more, 0.3 mm or more, 0.4 mm or more, or 0.5 mm or more. The range of the maximum particle size is, for example, 0.2 to 6 mm, 0.2 to 5 mm, 0.3 to 4 mm, or 0.5 to 3 mm. The maximum particle size referred to herein is a value set by, for example, passing the pulverized product through a sieve, and therefore may include a pulverized product having a size smaller than this value.

Unlike the juice-squeezing method, the extraction method using a solvent has a low rate of contamination with impurities such as cell fragments of the plant body, and the concentration of the actual active ingredients in the extract is high, such that medicinal effects can be obtained with a small amount of such an extract, which is particularly preferred.

### (2) Plant

The plant used as the raw material for preparing the plant extract constituting the agent for suppressing Aβ aggregation/deposition of one or more embodiments of the present invention is a plant belonging to the family Myrtaceae.

Examples of plants belonging to the family Myrtaceae include, but are not particularly limited to, plants of the genera Eucalyptus, Acca, Eugenia, Pimenta, Plinia, Psidium, and Myrtaceae. Specific examples thereof include Syzygium buxifolium, Syzygium samarangense, Syzygium jambos, feijoa, jabuticaba, pitanga, guava, Eucalyptus robusta, clove, and allspice. In particular, the genera Eucalyptus, Pimenta, Psidium, and Myrtaceae are preferred, with Myrtaceae being particularly preferred. Preferred species are guava, Eucalyptus robusta, clove, and allspice. The plant extract may be extracted from a plant as a whole belonging to the family Myrtaceae, or may be extracted from a part (organ) of the plant belonging to the family Myrtaceae. Examples of plant parts include leaves, stems, buds, leaf sheaths, petioles, aerial tubers, rhizomes, roots, seeds, hypocotyls, fruits, and flower buds. The plant extract can be extracted from any part (organ) of the plant, but it is preferred to extract the plant extract from the seeds and fruit of guava, from the leaves, stems, leaf sheaths, and petioles of Eucalyptus robusta, from the flower buds of clove, and from the fruit of allspice.

In particular, in the case of clove, the extract may be obtained by subjecting pulverized clove to solvent extraction such that the maximum particle size obtained by pulverizing clove is 6 mm or less, 5 mm or less, 4 mm or less, or 3 mm or less, and the lower limit of the maximum particle size is 0.1 mm or more, 0.2 mm or more, 0.3 mm or more, 0.4 mm or more, or 0.5 mm or more, and the pulverized product has a pulverized size (maximum particle size range) of, for example, 0.2 to 6 mm, 0.2 to 5 mm, 0.3 to 4 mm, or 0.5 to 3 mm. In this case, the solvents mentioned above can be used as preferred examples.

### 1-5-2. Effects

The agent for suppressing Aβ aggregation/deposition of one or more embodiments of the present invention comprises a plant extract that has been evaluated to exhibit potent Aβ aggregation/deposition suppression activity by the method of the above-described aspect 1-4. Specifically, the plant extract of one or more embodiments of the present invention exhibits a suppression rate (or aggregation/deposition suppression rate in the above-described 1-4) of 50% or more, 55% or more, 60% or more, 65% or more, 70% or more, 75% or more, 80% or more, 85% or more, 90% or more, 95% or more, 96% or more, 97% or more, or 98% or more, when compared to a control group to which no test substance is added, using the method of the above-described aspect 1-4.

The agent for suppressing Aβ aggregation/deposition of one or more embodiments of the present invention, which was searched for using the above evaluation method more similar to an in vivo environment using cerebral organoids, can exhibit a favorable effect of suppressing Aβ aggregation/deposition in actual body tissues (for example, the surface and/or inside of brain tissue cells in a living organism). The agent for suppressing Aβ aggregation/deposition of one or more embodiments of the present invention can exhibit an effect of suppressing a decline in/improving the cognitive function of a subject, which is the subject's function to recognize things via the brain, and thus can be used as an agent for suppressing cognitive function decline/improving cognitive function.

### 2. Composition for Suppressing Aβ Aggregation/Deposition

### 2-1. Outline

The second aspect of one or more embodiments of the present invention is a composition for Aβ aggregation/deposition suppression. The composition of the present invention comprises the agent for suppressing Aβ aggregation/deposition according to the first aspect as an active ingredient. According to the composition for aggregation/deposition suppression of one or more embodiments of the present invention, it becomes possible to apply the agent for suppressing aggregation/deposition in an easy-to-handle and stable state. This allows the composition to be provided in a form that is easier to administer, for example, as a pharmaceutical composition, by reducing the burden and invasiveness when administered in vivo (to the living organism).

### 2-2. Constitution

### 2-2-1. Constituent Factor

The composition for aggregation/deposition suppression of this aspect (hereinafter often abbreviated as “the present composition”) comprises an active ingredient as an essential constituent factor, and a solvent and/or carrier as optional constituent factors. Each of the constituent factors are specifically described below.

### (1) Active Ingredient

The composition of one or more embodiments of the present invention includes the agent for suppressing Aβ aggregation/deposition according to the first aspect as an active ingredient. The present composition may include one kind or two or more different kinds of agents for suppressing Aβ aggregation/deposition. Furthermore, other than the agent for suppressing Aβ aggregation/deposition according to the first aspect, known substances (nucleic acids, polypeptides, low molecular weight compounds, and the like) that have a same aggregation/deposition suppression action on Aβ may be selectively included.

The content of the agent for suppressing Aβ aggregation/deposition in the present composition varies depending on the type of the agent for suppressing Aβ aggregation/deposition contained and/or the effective amount thereof, the dosage form of the present composition, the type of carrier or additive described below, and the type of disease. Therefore, it can be determined appropriately, considering each condition.

As used herein, the term “effective amount” refers to the amount necessary for the agent for suppressing Aβ aggregation/deposition to exert its function as an active ingredient in the composition, and an amount that causes little or no harmful side effects in vivo to which it is applied. This effective amount may vary depending on various conditions such as the subject's information, route of administration, and number of administrations.

As used herein, the term “subject” refers to a living organism that is applicable for the agent for suppressing Aβ aggregation/deposition or the present composition. Examples thereof include humans, livestock (cows, horses, sheep, goats, pigs, chickens, ostriches, and the like), racehorses, pets (dogs, cats, rabbits, and the like), and laboratory animals (mice, rats, guinea pigs, monkeys, and the like). Humans (specifically referred to as “subjects” in this case) are preferred. Furthermore, the “subject's information” refers to various individual information about the living organism to which the suppressor or the present composition is applied, and in the case of a subject, includes, for example, the overall health condition, the progression and severity of a disease or illness if present, age, weight, sex, diet, drug sensitivity, whether or not concomitant medication is being used, and resistance to treatment. The effective amount of the agent for suppressing Aβ aggregation/deposition in the present composition and the dosage calculated based thereon are ultimately determined by the judgment of a physician, a veterinarian, or the like, depending on information about each individual subject and the like.

### (2) Solvent

The present composition may comprise a pharmaceutically acceptable solvent. A solvent is an optional component of the present composition and may be added as needed. The term “pharmaceutically acceptable” means that the solvent is harmless or has low toxicity to living organisms, can be generally used in the technical field of formulation, and may be used in pharmaceutical compositions.

Examples of the solvent include water or an aqueous solution, a buffer solution, an emulsifier, and an organic solvent.

Examples of pharmaceutically acceptable aqueous solutions include physiological saline, isotonic solutions containing glucose or other supplements, phosphate buffer, and sodium acetate buffer. Examples of supplements mentioned herein include D-sorbitol, D-mannose, D-mannitol, sodium chloride, as well as low-concentration nonionic surfactants and polyoxyethylene sorbitan fatty acid esters.

Examples of pharmaceutically acceptable organic solvents include ethanol.

### (3) Carrier

The present composition may comprise a pharmaceutically acceptable carrier. A carrier is an optional component of the present composition and may be added as needed.

Examples of carriers include suspending agents, diluents, solubilizing agents, dispersing agents, surfactants, emulsifying agents, soothing agents, stabilizers, preservatives, antiseptics, antioxidants, buffers, and isotonic agents.

In addition to the above, if necessary, the present composition may contain, as appropriate, an excipient, a filler, a binder, a disintegrant, an absorption enhancer, a bulking agent, a moisturizing agent, a humectant, an adsorbent, a disintegration suppressor, a coating agent, a colorant, and the like that are commonly used in pharmaceuticals.

Such carriers are primarily used to facilitate formulation and maintain the dosage form and drug efficacy, as well as to make the active ingredient, the agent for suppressing Aβ aggregation/deposition, less susceptible to degradation in vivo, and may be used appropriately as needed.

### 2-2-2. Dosage Form and Administration Method

The dosage form of the present composition is not particularly limited as long as it does not inactivate the active ingredients, such as the agent for suppressing Aβ aggregation/deposition and other known agents for suppressing Aβ aggregation/deposition, and is capable of exerting the pharmacological effects of the active ingredients in vivo after administration. The specific dosage form of the present composition may be appropriately selected depending on the administration method and/or prescription conditions. In general, administration methods can be roughly divided into oral administration and parenteral administration, and the present composition may be prepared in a dosage form suitable for each administration method.

For oral administration, examples of dosage forms include solid preparations (including tablets, pills, sublingual tablets, capsules, and drops), granules, powders, dispersing agents, and liquid preparations (including oral solutions, suspensions, emulsions, and syrups). The solid preparations may be in the form of coated tablets known in the art, such as sugar-coated tablets, gelatin-coated tablets, enteric-coated tablets, film-coated tablets, double tablets, and multi-layer tablets, if necessary. Oral administration is the preferred method of administration as it is minimally invasive and easy to administer.

Parenteral administration can be divided into systemic administration and local administration, and local administration can be further subdivided into intratissue administration, transepidermal administration, transmucosal administration, and rectal administration. The present composition may also be prepared in a dosage form suitable for each administration method. For example, dosage forms suitable for systemic or intratissue administration include injections, which are liquid preparations. Dosage forms suitable for transepidermal or transmucosal administration include liquid preparations (including liniments, eye drops, nasal drops, and inhalants), suspensions (including emulsions and creams), powders (including nasal drops and inhalants), pastes, gels, ointments, and plasters. Examples of dosage forms suitable for rectal administration include suppositories.

The method for administering the present composition varies depending on the type of applicable disease and the applicable area, as described below. There are no limitations on the administration method, as long as the agent for suppressing Aβ aggregation/deposition, the active ingredient of the present composition, can ultimately reach the applicable area and exert its effect. For example, in a case in which the applicable disease is a neurodegenerative disease, the applicable area is mainly the cerebrospinal area, which is part of the central nervous system. In this case, the administration method may be intratissue administration, i.e., direct administration into the central nervous system, or systemic administration via the circulatory system. Specific examples include intratissue administration by intracerebrospinal injection, or intracirculatory administration such as intravascular injection (including intravenous injection and intraarterial injection) or intralymphatic injection. In general, direct administration, such as intratissue administration, can reliably deliver active ingredients to the applicable area at high concentrations. Meanwhile, systemic administration via the circulatory system allows delivery to the applicable area in a minimally invasive manner and without leakage. The method to be selected can be determined appropriately depending on the situation and pathological condition.

Injections may be formulated by appropriately combining the above-described emulsifiers, suspending agents, surfactants, stabilizers, pH adjusters, and the like, and mixing them in a unit dose form required by generally accepted pharmaceutical practice, and are provided in the form of unit dose ampoules or multi-dose containers.

The specific shape and size of each of the above dosage forms are not particularly limited as long as they are within the range of dosage forms known in the art.

### 2-2-3. Applicable Disease

The present composition can be applied to treat or prevent Alzheimer's dementia and mild cognitive function impairment caused by Aβ aggregation/deposition.

Alzheimer's dementia is developed as follows: amyloid β proteins are aggregated and accumulated in neurons in the brain, microtubule-binding proteins, tau proteins, are hyperphosphorylated to become fibrotic, neurons are destroyed, and the brain is then shrunk. Therefore, the aggregating proteins that cause Alzheimer's dementia may correspond to amyloid β proteins, tau proteins, α-synuclein, and the like. The agent for suppressing Aβ aggregation/deposition contained in the present composition can be effective in suppressing the progression of Alzheimer's dementia or preventing Alzheimer's dementia by suppressing Aβ aggregation/deposition.

Meanwhile, mild cognitive function impairment is a cognitive impairment intermediate between that of a healthy individual and that of an individual with dementia, and is characterized by a mild decline in cognitive function. The most common cause of mild cognitive function impairment is Alzheimer's dementia. Therefore, the present composition can also be used to treat or prevent mild cognitive function impairment.

### 2-3. Effects

The present composition can be effectively used for the treatment or prevention of Alzheimer's dementia or mild cognitive function impairment.

### 3. Agent for Suppressing Decline in/Improving Cognitive Function in Subject

### 3-1. Outline

The third aspect of one or more embodiments of the present invention is an agent for suppressing a decline in/improving the cognitive function of a subject, which is the subject's function to recognize things via the brain. Like the agent for suppressing Aβ aggregation/deposition according to the first aspect, the agent for suppressing cognitive function decline/improving cognitive function in a subject of one or more embodiments of the present invention contains an extract of a plant belonging to the family Myrtaceae (particularly, clove or allspice) as an active ingredient.

### 3-2. Constitution

### 3-2-1. Constituent Factor

The agent for suppressing cognitive function decline/improving cognitive function in a subject of this aspect comprises an active ingredients as an essential constituent factor, and a solvent and/or carrier as optional constituent factors.

Like the agent for suppressing Aβ aggregation/deposition according to the first aspect, the agent for suppressing cognitive function decline/improving cognitive function in a subject of this aspect may comprise an extract of a plant belonging to the family Myrtaceae as an active ingredient. In addition, other than the extracts of a plant belonging to the family Myrtaceae, known substances (nucleic acids, polypeptides, low molecular weight compounds, and the like) that have a same action of suppressing cognitive function decline/improving cognitive function in a subject may be selectively included.

The content of the extract of a plant belonging to the family Myrtaceae in the agent for suppressing cognitive function decline/improving cognitive function in a subject of this aspect varies depending on the type of the extract of a plant belonging to the family Myrtaceae contained and/or the effective amount thereof, the dosage form, the type of carrier or additive, and the type of disease. Therefore, it can be determined appropriately, considering each condition.

The “effective amount,” “subject,” “solvent,” and “carrier” may be the same as those explained in the composition according to the second aspect above.

### 3-2-2. Dosage Form and Administration Method

The dosage form (formulation) and administration method of the agent for suppressing cognitive function decline/improving cognitive function in a subject of this aspect may be the same as those described for the composition according to the second aspect above.

### 3-3. Effects

According to the agent for suppressing cognitive function decline/improving cognitive function in a subject of this aspect, cognitive function decline can be prevented, and cognitive function can be improved.

### EXAMPLES

Hereafter, one or more embodiments of the present invention are described in greater detail with reference to Examples, although the technical scope of the present invention is not limited to these Examples.

The reagents used in the Examples were as follows:

### <Reagents>

STEMdiff (trademark) Neural Progenitor Medium (NPM) (STEMCELL/5833)
Neuro basal medium minus phenol red (Gibco/12348017)
hESC-Qualified Matrigel (hES-matrigel) (Corning/354277)
Accutase (STEMCELL/7920)
DMEM/F-12 (Gibco/21041025)
Y-27632 (nacalai/08945-84)
PBS (Gibco/10010-031)
B-27 (Gibco/17504044)
Penicillin-Streptomycin (Nacalai/26253-84)
Amyloid β-Protein human 1-42 (Peptide Institute, Inc./4349-v)
1,1,1,3,3,3-Hexafluoro-2-2propanol (HFIP) (Tokyo Chemical Industry Co., Ltd. (TCI)/H0424)
Dimethyl sulfoxide (DMSO) (FUJIFILM Wako Pure Chemical Corporation/046-21981)
QD reagent (described below)
Plant material solution (100 mg/ml solution: described below)
Physiological saline (PS) (Otsuka Pharmaceutical Co., Ltd.)
1-ml syringe
0.2-μm filter
Preparation of NbM medium

NbM medium was prepared by adding 1 ml of B-27 (Gibco/17504044) and 500 μl of Penicillin-Streptomycin (Nacalai/26253-84) to 50 ml of Neurobasal Medium, minus phenol red (Gibco/12348017).

### [Example 1] Preparation of Cerebral Organoids

### (1) Culture and Passaging of iPS Cells

The human iPS cells used were HPS08554 (RIKEN BRC). The human iPS cells were seeded onto a cell culture dish coated with Matrigel (Corning), Vitronectin (Life Technologies Japan Ltd.), iMatirx (Takara Bio Inc., T303), or fibronectin (Recombinant Human Fibronectin GMP Protein, CF Summary). The culture medium used for maintenance was mTeSR1 (STEMCELL Technologies), TeSR2 (STEMCELL Technologies), Essential 8 (trademark) (Life Technologies Japan Ltd.), or StemFit.

In the case of culture on Matrigel or Vitronectin, Accutase (Life Technologies Japan Ltd.) or GCDR (STEMCELL Technologies) was used, in the case of culture on fibronectin, a 0.05% ethylenediaminetetraacetic acid (EDTA) solution was used, and in the case of culture on iMatirx, TrypLE Select (Life Technologies Japan Ltd.) or TrypLE Express was used, as a cell detachment agent during passaging.

In addition, Y-27632 (Wako Pure Chemical Industries, Ltd.) was added to the medium to a concentration of 10 μM only at the time of cell seeding. The medium was exchanged every other day or every day. Human iPS cells with up to 50 passages were used in the experiment.

### (2) Preparation of Neural-Induced Embryoid Bodies (EBs)

After thawing, human iPS cells that had been passaged at least twice were treated with the detachment agent for 5 to 15 minutes to detach them from the culture substrate, and then dispersed into single cells by pipetting using a pipette. These single cells were suspended in STEMdiff (trademark) Neural Induction Medium containing Y-27632 (Wako Pure Chemical Industries, Ltd.) at a final concentration of 10 μM, supplemented with STEMdiff (trademark) SMADi Neural Induction Supplement (hereinafter referred to as “NIMS medium”). The cells were seeded onto a PLO/Laminin coating or Matrigel (registered trademark) (Corning (registered trademark)) and cultured in an environment of 5% CO₂ and 37 °C.

After confirming cell adhesion, the entire amount of NIMS medium was exchanged every day. On day 7, the adherent cells were detached with Accutase, and the detached cells were expanded and cultured in STEMdiff (trademark) Neural Progenitor Medium on a PLO/laminin coating or Matrigel for three passages. The cells were suspended in an NbM plus Shh medium containing a mixed medium of 50 ml of Neuro basal Medium minus phenol red (Thermo Fisher Scientific) supplemented with 1 ml of B-27 supplement (Thermo Fisher Scientific) and Penicillin-Streptomycin Mixed Solution (NACALAI TESQUE, INC.) (hereinafter referred to as “NbM medium”), and human recombinant sonic hedgehog (SHH) (VERITAS Corporation) adjusted to a final concentration of 1 ng/ml, thereby preparing a cell suspension of 4×10⁵ cells/ml. The suspension was seeded into a U-bottom 96-well plate at 0.1 ml per well, and cultured for 24 hours in an environment of 5% CO₂ and 37 °C, thereby preparing neural-induced EBs.

Alternatively, human iPS cells cultured in mTeSR1 (STEMCELL Technologies) were treated with GCDR to dissociate into single cells, thereby preparing a cell suspension of 9×10⁴ cells/ml, and 0.1 ml of the suspension per a well was seeded into a 96-well U-bottom plate. The medium was exchanged every two days using an EB Formation medium provided in a STEMdiff (trademark) Cerebral Organoid Kit, and the cells were cultured for five days in an environment of 5% CO₂ and 37 °C. The medium was then exchanged with an induced medium, and the EBs formed from the iPS cells were cultured for additional two days to induce neural induction, thereby preparing neural-induced EBs.

### (3) Gel-Embedded Culture of Neural-Induced EBs

EBs 2 days after addition of the hedgehog signal (Day 2) and 5 days after addition of the hedgehog signal (Day 5) were carefully collected using a wide-mouth tip without disrupting the EBs, and 12 EBs were embedded in Matrigel. There are several embedding methods, but in this Example, parafilm was attached to a 48-well plate; the required number of depressions were created using the chip attachment part, 12 EBs were placed in the depressions, and excess medium was removed. Then, 15 μl of Matrigel was added dropwise and left to stand in an incubator for 0.5 to 2 hours. A fresh NbM medium was then added dropwise to a 6-well floating plate, and the EBs were collected using a tip of which the pointed end had been cut with scissors without disrupting the Matrigel.

Each EB embedded in Matrigel was cultured in an environment of 5% CO₂ and 37 °C on a bioshaker while being rotated at 90 rpm. During the gel-embedded culture period, half of the NbM medium was exchanged every three days.

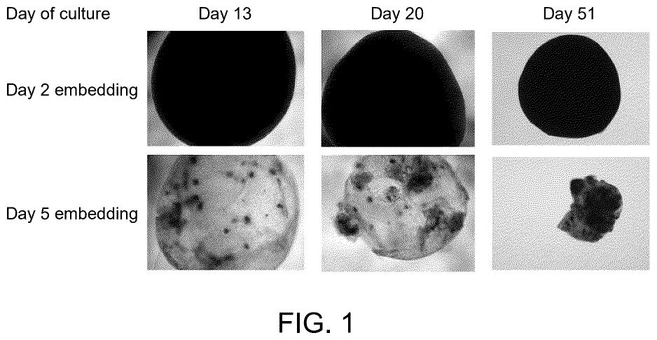

As shown in FIG. 1, as a result of confirming the formation of organoid-like tissue, a favorable tissue was formed in the EBs from Day 2, whereas in the EBs from Day 5, the cells did not sufficiently infiltrate or fill the gel.

### (4) Gel-Embedded Culture and Quantification of CCL2 in Culture Supernatant

Each EB embedded in Matrigel was cultured in an environment of 5% CO₂ and 37 °C using a bioshaker while rotated at 90 rpm. Cells within the gel actively infiltrate and grow, thereby filling the gel space. Therefore, the culture medium was collected from the early stage of gel culture (unfilled stage: D0), the growth stage of gel culture (unfilled stage: D7), the cell-filled stage of gel culture (filled stage: D14), and the cell-filled stage of gel culture (filled stage: D21), and the amount of cytokine secretion (CCL2) in the culture supernatant was quantified by ELISA.

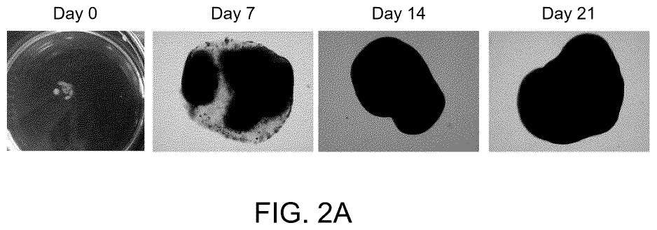

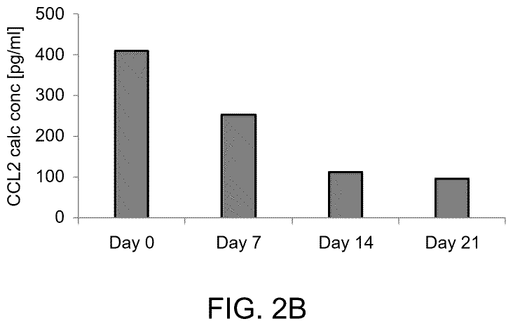

The results are shown in FIG. 2. It was found that there was a correlation between the cell infiltration status within the gel (FIG. 2A) and the amount of CCL2 (monocyte chemotactic protein) secreted (FIG. 2B). It was revealed that the amount of CCL2 secreted remained high while the cells were infiltrating and filling the gel, and then decreased as filling progressed, eventually converging to a constant level.

In this way, by measuring the amount of CCL2 in the culture supernatant during the organoid-like tissue formation step, it is possible to confirm whether cells have filled inside the gel.

### [Example 2] Preparation of Organoids for Visualizing Deposition of Aggregating Proteins, and Method for Searching for Agent for Suppressing Aggregation/Deposition of Aggregating Proteins Using the Organoids

The end of a 1000-μl tip was cut off with sterilized scissors, and the organoids produced in Example 1 that had grown to a diameter of 1 mm or more were aspirated without disrupting them, and transferred to a U-bottom 96-well plate. Then, the medium was removed from the U-bottom 96-well plate, and 25 μl of a quantum dot-modified amyloid β40 protein (QD-Aβ₄₀) solution (i), 25 μl of an amyloid β₄₂ protein (Aβ₄₂) solution (ii), and 50 μl of a test specimen solution (iii) (ratio of solution (i):solution (ii):solution (iii)=1:1:2) were added dropwise to each well, and the plate was incubated at 37 °C in 5% CO₂ for 24 hours (final concentration: QD-Aβ₄₀: 30 nM; artificial Aβ₄₂ solution: 10 μM; test specimen (rosmarinic acid: 50 μM)).

Each solution was prepared as follows.

### (i) Preparation of QD-Aβ₄₀ Solution (120 nM)

Aβ₄₀ was modified with a quantum dot (QD) in the manner described below to prepare a quantum dot-modified amyloid β protein (QD-Aβ₄₀).

Qdot (QD) (trademark) 605 ITKTM amino (PEG) Quantum Dots (8 μM, 125 μl) were introduced into two 1.5-ml tubes, and the tubes were centrifuged at 10,000×g and 4 °C for 1 minute. The supernatants thereof were transferred to centrifugal tubes, and 4,500 μl PBS was added thereto. Centrifugation was continued at 4 °C and 3,800× g until the total amount in the two tubes was reduced to about 50 μl or less, and the filtrates were discarded. After PBS was filled, centrifugation was performed again until the amount was reduced to 50 μl. The QD solutions obtained were introduced into a tube to adjust the total amount to approximately 180 μl. 10 mM sulfo-EMCS (20 μl) was added, and the resultant was allowed to stand at room temperature for 1 hour to prepare QD-EMCS. After the preparation of QD-EMCS, 20 μl of 100 mM K-glutamic acid was added in order to inactivate an unreacted N-hydroxysuccinimide group contained in the solution, and the resultant was allowed to stand at room temperature for 10 minutes.

Two columns for desalting were prepared, approximately 800 μl of resin was introduced into each thereof, and centrifugation was performed at 1,000×g and 4 °C for 1 minute. PBSE (300 μl) was loaded onto each column and centrifugation was performed at 1,000×g and 4 °C for 1 minute. This procedure was performed two times to prepare desalting columns.

The QD-EMCS (110 μl) was allowed to impregnate into the center of each of two desalting columns, and 15 μl of PBSE was added as a stacker. After the columns were subjected to centrifugation at 1,000×g and 4 °C for 2 minutes, filtrates (desalted QD-EMCS) of the two desalting columns were gathered together. To the desalted QD-EMCS obtained, 20 μl of 1.0 mM Cys-Aβ/DMSO were added and mixed therein, and the resulting mixture was then allowed to stand at room temperature for 1 hour. Thereafter, 20 μl of 100 mM 2-mercaptoethanol was added in order to inactivate an unreacted maleimide group contained in the solution, and the resultant was allowed to stand at room temperature for 10 minutes.

Using the above procedure, the filtrate was transferred in an amount of 145 μl each to two centrifugal concentration tubes, 4,500 μl of water was added thereto, and the resultants were centrifuged at 3,800×g and 4 °C for 17 minutes. The filtrates were discarded, and the obtained solutions were introduced into a tube to adjust the total amount of the obtained solutions to approximately 140 μl.

Water (300 μl) was introduced into a desalting column, centrifugation was performed at 1,000×g and 4 °C for 1 minute, and this procedure was performed two times. The filtrate (70 μl) was allowed to impregnate the desalting column, and 15 μl of stacker water was added. Centrifugation was performed at 1,000×g and 4 °C for 2 minutes to obtain QD-Aβ₄₀ of interest.

The synthesized QD-Aβ₄₀ solution (80 μM) was diluted with NbM medium to 120 nM and treated in an ultrasonic cleaner at 25 °C and 43 kHz for 5 minutes.

### (ii) Preparation of Artificial Aβ₄₂ Solution (40 μM)

To a reagent bottle containing 0.5 mg of Aβ₁₋₄₂ (manufactured by Peptide Institute, Inc.; Cat No. 4394v), 500 μl of 1,1,1,3,3,3-hexafluoro-2-propanol (HFIP) solution was added. After pipetting, the reagent bottle was sealed with parafilm and left to stand at room temperature in a clean bench for 1 hour. The lid of the reagent bottle was then opened in the clean bench to allow the HFIP to evaporate. After confirming that all HFIP had evaporated, 107 μl of DMSO was added and dissolved over 15 minutes. The solution was dispensed in 10-μl aliquots into tubes and stored frozen at −80 °C. This cryopreserved 1 mM Aβ₄₂ solution was thawed, pipetted 20 times or more, and diluted 25-fold with NbM medium, thereby preparing a 40 μM Aβ₄₂ solution.

(iii) Preparation of Test Specimen-Containing Solution

### Preparation of Low-Molecular-Weight Compound Solution (100 μM)

Rosmarinic acid (hereinafter referred to as “RA”) was dissolved in DMSO to prepare a 1 mM RA solution, which was then diluted with NbM medium, thereby preparing a 100 μM RA solution.

In addition, 1 μl of DMSO solution and/or 150 mM physiological saline was diluted with 19 μl of NbM, and 3 μl of this diluted solution was further withdrawn and diluted with 72 μl of NbM, thereby preparing a solution.

The next day, all of the culture supernatant was removed without aspirating the organoid, and 150 μl of fresh NbM medium was added. The cultured organoids were then observed and photographed under a fluorescence microscope.

After photographing, the organoids were returned to the incubator and cultured again, and observation and photographing were carried out under the fluorescence microscope in the same manner from the next day onwards.

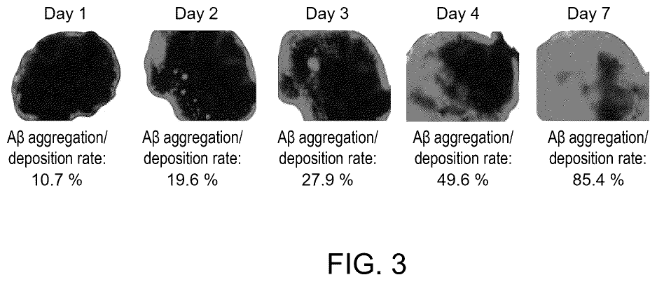

FIG. 3 shows the time course of Aβ aggregation and deposition observed and photographed.

As shown in FIG. 3, it was confirmed that the organoids for visualizing the deposition of aggregating proteins could be observed for at least 8 days, with the deposition amount of aggregating proteins gradually increasing as the number of culture days increased, while the organoids remained viable. These results demonstrate that it is possible to create organoids for visualizing the deposition of aggregating proteins, which mimics the rate at which aggregating proteins accumulate with aging in the human brain.

### [Example 3] Method for Quantifying Deposition of Aggregating Proteins in Organoids

Using a fluorescence microscope (BZ-X800 manufactured by KEYENCE CORPORATION), the cultured organoids in Example 2 were photographed in a bright field at a high resolution with an exposure time of 1/500 s to 1/2000 s, or at a high sensitivity with an exposure time of 1/2000 s to 1/5000 s, with Z stacks with a pitch of 20 μm. When a ×2 objective lens was used, one image showing the organoids as a whole was photographed, or when a ×4 objective lens was used, one image with the center of the cultured organoids as the center position, and three images including the outline of the cultured organoids were photographed. A total of four images were obtained. Subsequently, to observe the deposition of quantum dot-modified amyloid β in the cultured organoid, red fluorescence was observed using a TRITC lens at a high sensitivity with an exposure time of ¼ s to 1/8.5 s, and images were obtained according to the procedure described above.

Each of the images obtained above was analyzed as follows. Specifically, the organoid images used in various tests were loaded using the hybrid cell counting function, the bright field was selected, and the extraction selection was set to OFF. The threshold of the loaded bright-field image was set to 200, and the organoid area (organoid surface area) was calculated.

Next,
an image of organoids to which QD-Aβ₄₀/artificial Aβ₄₂ was added during organoid culture without the addition of a test specimen;
an image of organoid as a negative control in which DMSO and/or physiological saline were added as a test specimen to QD-Aβ₄₀/artificial Aβ₄₂; and
an image of organoids to which rosmarinic acid (RA) was added as a test specimen
   were loaded.

The black balance level of image processing was adjusted for the fluorescent images of various tests. First, the negative control image was read out. The initial value for images taken with a ×2 objective lens was [50], and that for images taken with a ×4 objective lens was [70]. It was confirmed whether the fluorescent region of the aggregating proteins deposited in the organoids could be separated from the background. When the fluorescent region of the aggregating proteins deposited in the organoids could not be separated from the background even at the above black balance level, the black balance level was varied in 5-unit increments until the optimal black balance level was set.

After combining the fluorescent image after applying the black balance and the bright-field organoid image, fluorescence was selected using the hybrid cell count function, and one extraction was set. The advanced mode for brightness mode was selected, the extraction setting range was set to 0-299. After selecting organoids, the blur filter was unchecked, the threshold was set to 25, and the area of the fluorescent range of the aggregating proteins (deposition area of aggregating proteins) was calculated.

The aggregation/deposition rate (%) of aggregating proteins in organoids was calculated using the following formula (1):

Aggregation/deposition rate of aggregating proteins in organoids(%)=(Aggregation/deposition area of aggregating proteins/organoid surface area)×100 (1).

In addition, the condition for the test system to be established was that the aggregation/deposition rate (%) of aggregating proteins was in a range of 90% to 100% when DMSO and/or physiological saline was added.

Furthermore, when this test system was established, by setting up a group to which the test specimen was also added simultaneously, it was possible to quantitatively analyze the effect of the specimen on the suppression of aggregation/deposition of aggregative proteins in organoids. Specifically, the aggregating protein aggregation/deposition suppression function of the test specimen could be quantified as the aggregation/deposition suppression rate (%), which could be calculated using the following Formula (2):

Aggregating protein(Aβ in this Example)aggregation/deposition suppression rate (%)=(Aggregating protein aggregation/deposition rate in the absence of test substance(%))−(Aggregating protein aggregation/deposition rate in the presence of test substance(%)) (2).

It can be seen that the larger the aggregation/deposition suppression rate (%) calculated by Formula (2), the greater the aggregation/deposition suppression effect of the test specimen. This evaluation technique can be used to select drugs, compounds, foods, and substances that can prevent or delay the deposition of aggregating proteins in cells or tissues.

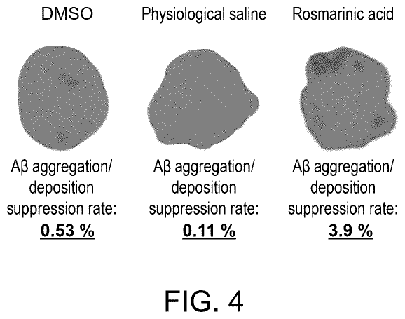

The results of the Aβ aggregation/deposition suppression effect of each test specimen in this Example are shown in FIG. 4.

As shown in FIG. 4, the Aβ aggregation/deposition suppression rate was calculated 24 hours after the addition of each drug: DMSO, physiological saline, or rosmarinic acid. As a result, the rate of Aβ aggregation/deposition suppression in cerebral organoids was 0.53% for DMSO, the rate of Aβ aggregation/deposition suppression in cerebral organoids was 0.11% for physiological saline, and the rate of Aβ aggregation/deposition suppression in cerebral organoids was 3.9% for rosmarinic acid. These results revealed that rosmarinic acid, which is known to have an effect of suppressing Aβ aggregation, is not a drug that has an effect of suppressing Aβ aggregation/deposition in cerebral organoids.

### [Example 4] Search for Amyloid β (Aβ) Aggregation/Deposition Inhibitor

Using the quantitative system for Aβ aggregation/deposition in cerebral organoids constructed as described above, the present inventors searched for materials (plant extracts) that have an effect of suppressing Aβ aggregation/deposition.

### (i) Preparation of Test-Specimen-Containing Solution

Guava seeds and fruits; leaves, stems, leaf sheaths, and petioles of Eucalyptus robusta; clove flower buds; and allspice berries (all from the family Myrtaceae); leaves, stems, leaf sheaths, petioles, and petals of Lantana montevidensis; and leaves, stems, leaf sheaths, and petioles of sweet Mexican herb (all from the family Verbenaceae); and leaves, stems, leaf sheaths, and petioles of Glechoma hederacea var. grandis (all from the family Lamiaceae) were used as test specimens (final extract concentration at the time of testing: 0.01 mg/well).

Each test specimen was washed, dried, and pulverized to a size of 5 mm or less in length×5 mm or less in width. 90% EtOH (30 ml) was added to 1 g of each pulverized product, and the mixture was extracted by shaking at 150 rpm at 28 °C in the dark for 20 hours. The extract liquid was centrifuged at 3,000 rpm for 20 minutes, filtered through a cotton plug, concentrated by centrifugation, and then lyophilized. The dried product was dissolved in DMSO. Thus, 100 mg/ml of various plant extracts were prepared and stored at −20 °C until use.

Each of these plant extracts was diluted 25-fold with NbM medium. The diluted solution (40 μl) was diluted with 960 μl of NbM medium and sterilized by filtration through a 0.2 μm filter, thereby preparing a solution containing 0.02 mg of the material.

### (ii) Measurement of Aβ Aggregation/Deposition Suppression Activity

The effect of each plant extract on the suppression of Aβ aggregation/deposition in cerebral organoids was measured at a final extract concentration of 0.01 mg/well. The cerebral organoids, reagents including QD-Aβ, operating procedures, and a method for image processing to a method for calculation of the aggregation/deposition suppression rate used were as described in Examples 1 to 3 above. DMSO and rosmarinic acid were used as comparison groups.

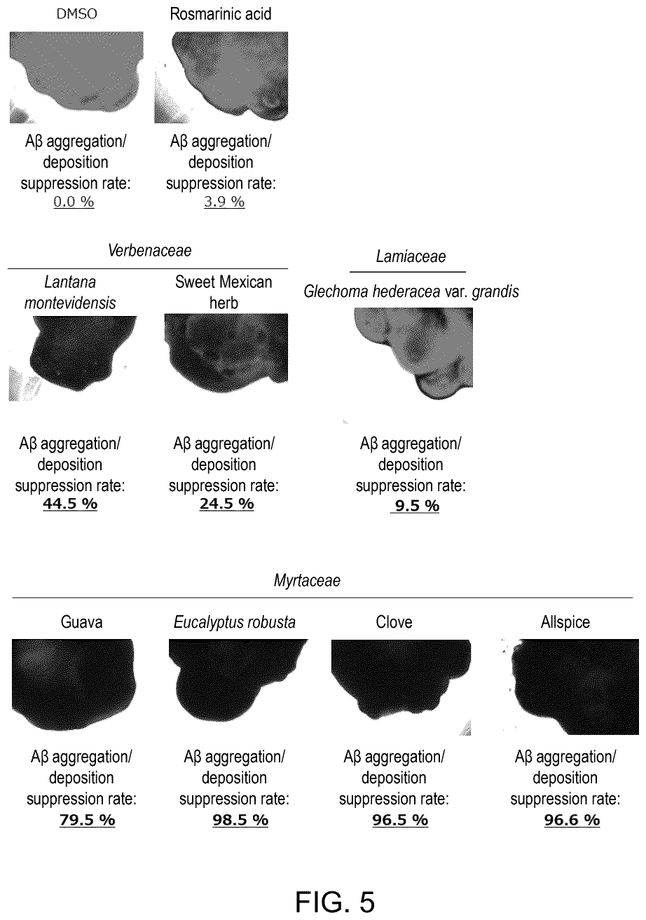

The results of observation and photography of the effects of various test specimens on the suppression of aggregation/deposition of aggregating proteins are shown in FIG. 5. Table 2 also shows the Aβ aggregation/deposition suppression rate (%) 24 hours after the addition of the specimens.

TABLE 2

| | | Aβ aggregation/deposition |
|---|---|---|
| Test specimen | | suppression rate (%) 24 hours |
| (plant) | Family | after the addition of specimens |
| Guava | Myrtaceae | 79.5 |
| Eucalyptus robusta | Myrtaceae | 98.5 |
| Clove | Myrtaceae | 96.5 |
| Allspice | Myrtaceae | 96.6 |
| Lantana | Verbenaceae | 44.5 |
| montevidensis | | |
| Sweet Mexican herb | Verbenaceae | 24.5 |
| Glechoma hederacea | Lamiaceae | 9.5 |
| var. grandis | | |

The results shown in FIG. 5 and Table 2 demonstrated that the plant extract of the family Myrtaceae has an excellent effect of suppressing Aβ aggregation/deposition on the surface of and/or inside a cerebral organoid.

### [Example 5] Evaluation of Pulverized Clove Size

The relationship between the pulverized clove size and the Aβ aggregation/deposition suppression rate was evaluated.

### (i) Preparation of Pulverized Clove Product

Clove was pulverized by a mill, thereby obtaining the following pulverized clove products.
Pulverized product A: Pulverized size of 5 mm or less (the maximum particle size of the particles contained in the pulverized product is 5 mm or less)
Pulverized product B: Pulverized size of 2 mm or less (the maximum particle size of the particles contained in the pulverized product is 2 mm or less)
Pulverized product C: Pulverized size of 0.5 mm or less (the maximum particle size of the particles contained in the pulverized product is 0.5 mm or less)

In other words, all particles contained in pulverized product A have a particle size of 5 mm or less, all particles contained in pulverized product B have a particle size of 2 mm or less, and all particles contained in pulverized product C have a particle size of 0.5 mm or less.

### (ii) Preparation of Test Specimen-Containing Solution

90% EtOH (30 ml) was added to 1 g of each pulverized product in (i) above, and the mixture was extracted by shaking at 150 rpm at 28 °C in the dark for 20 hours. The extract liquid was centrifuged at 3,000 rpm for 20 minutes, filtered, concentrated by centrifugation, and then lyophilized. The dried product was dissolved in DMSO. Thus, 100 mg/ml of various plant extracts were prepared and stored at −20 °C until use. The yield of a pulverized product A extract was 13.7%, the yield of a pulverized product B extract was 17.4%, and the yield of a pulverized product C extract was 21.4%. Therefore, the smaller the pulverized size, the higher the yield.

Each of these plant extracts was diluted 25-fold with NbM medium. The diluted solution (40 μl) was diluted with 960 μl of NbM medium and sterilized by filtration through a 0.2 μm filter, thereby preparing a solution containing 0.02 mg of the material.

(iii) Measurement of Aβ Aggregation/Deposition Suppression Activity

The measurement was carried out in the same manner as in Example 4.

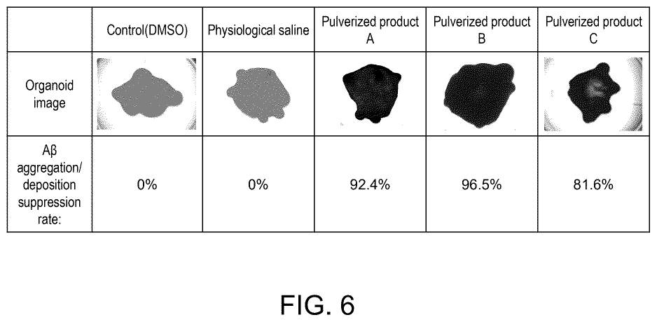

The results of observation and photography of the effects of various test specimens on the suppression of aggregation/deposition of aggregating proteins are shown in FIG. 6. Table 3 also shows the Aβ aggregation/deposition suppression rate (%) of each test specimen.

TABLE 3

| | Aβ aggregation/deposition |
|---|---|
| Test specimen | suppression rate (%) |
| DMSO | 0% |
| Physiological saline | 0% |
| Plant extract extracted from pulverized | 92.4% |
| product A | |
| Plant extract extracted from pulverized | 96.5% |
| product B | |
| Plant extract extracted from pulverized | 81.6% |
| product C | |

In FIG. 6, the area where red fluorescent quantum dot-modified amyloid β was deposited exhibits red fluorescence. As shown in FIG. 6 and Table 3, it was revealed that each of the plant extracts prepared from pulverized products A, B, and C has a high Aβ aggregation/deposition suppression activity. This demonstrates that it is preferable to extract a plant extract from a pulverized product having a pulverized size of 5 mm or less, 2 mm or less, or 0.5 mm or less, with a maximum particle size of around 2 mm being particularly preferable. Interestingly, it was found that from the comprehensive viewpoint of extraction recovery rate and Aβ aggregation/deposition suppression rate, the preferred pulverized size (maximum particle size range) is in a range of 5 mm to 0.5 mm and a range of 3 mm to 0.5 mm. Furthermore, other study results also demonstrate that the range of 3 mm to 0.5 mm is particularly preferable. It was revealed that controlling the pulverized size is extremely important for achieving high extraction recovery rates and high Aβ aggregation/deposition suppression rates.

### [Example 6] Evaluation of the Effects of Plant Extracts on Preventing Cognitive Function Decline/Improving Cognitive Function

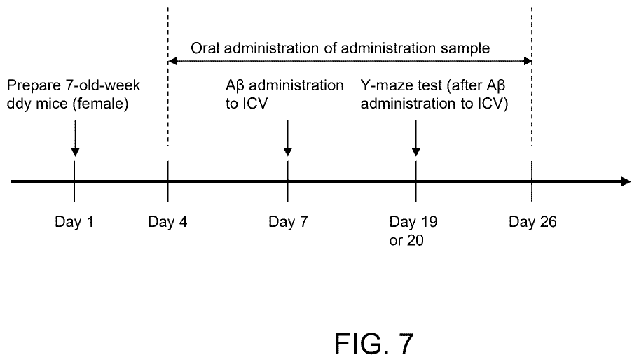

The effects of allspice extract and clove extract on preventing cognitive function decline/improving cognitive function were evaluated using mice. The evaluation schedule is shown in FIG. 7.

### (i) Animal Rearing

Seven-week-old ddY mice (female) were prepared. This day was designated as the first day after the mice arrived (first day of rearing). For each sample, 26 animals were randomly assigned to cages of four each.

### (ii) Preparation of Aβ₄₂ for Administration to Mice

Aβ₄₂ (Peptide Institute, Inc.: Code 4394) (5 mg) was added to 2.3 ml of hexafluoro-2-propanol (HFIP) and dissolved therein at room temperature for 30 minutes. The resulting solution was dispensed into tubes in 100 μl aliquots while cooling on ice and then dried. The amount of Aβ per tube was 47810 μmol=215.81 μg/100 μl HFIP/tube. These Aβ tubes were stored in a deep freezer at −60 °C

(iii) Advance Preparation Before Intracerebroventricular Administration (ICV Administration)

A two-stage tuberculin 3 m/m manufactured by Natsume Seisakusho Co., Ltd. was attached to a microsyringe manufactured by HAMILTON. 6 mM NaOH (64.6 μl) was added to the Aβ tubes prepared in the previous section, and the mixture was ultrasonically stirred in a room temperature water bath for 10 minutes to dissolve Aβ. The dissolved Aβ was dispensed into 1.5-ml Eppendorf tubes in 32.3 μl aliquots. 10×PBS (34.5 μl) was taken into a new Eppendorf tube, 279.4 μl of Milli-Q was added, and the mixture was vortexed. The resulting solution (142.7 μl) was then taken out and dispensed into the Eppendorf tubes containing 32.3 μl of Aβ. In this way, microsyringes containing Aβ dissolved in 1×PBS were prepared.

### (iv) Preparation of the Administration Samples

Gum arabic was added to various plant extracts (allspice extract, clove extract) and ground in a mortar such that gum arabic was adjusted to 2%. Each sample was then dissolved with the addition of physiological saline so as to be adjusted to 500 mg sample/kg body weight/2% gum arabic/10 ml physiological saline. The prepared administration samples were dispensed into 4.5-ml tubes at daily amounts to be used and stored in a deep freezer. An administration sample as a control (physiological saline) was also adjusted at the same time to 0 mg/kg body weight/2% gum arabic/10 ml physiological saline. At the time of administration, each administration sample was taken out from the deep freezer, placed in a water bath at 30 °C, and dissolved by shaking.

### (v) Preparation of Mice for Oral Administration of Allspice and Clove Extracts

The administration samples of allspice and clove extracts prepared in (iv) were orally administered to the mice at a dose of 500 mg/kg per mouse from day 4 to day 26 after the mice arrived.

### (vi) Preparation of Mice for Oral Administration of Control

The control (physiological saline) administration sample was orally administered to the mice at the same dose per mouse as described above from day 4 to day 26 after the mice arrived.

For the mice in both (v) and (vi), oral administration was performed from rearing day 4 to day 26 after the mice arrived, and the sample administered was started before the model mice were produced.

(vii) Production of Model Mice by Intracerebroventricular (ICV) Administration of Aβ (Hereafter Referred to as ICV-Aβ) (Mouse Rearing: Day 7)

On rearing day 7 after the mice arrived, the mice for oral administration of allspice extract, the mice for oral administration of clove extract, and the mice for oral administration of physiological saline were anesthetized by inhalation using sevoflurane. The needle tip was aligned vertically at a position 0.3 mm posterior, 1.1 mm lateral, and 4 mm ventral from the bregma of each mouse, and 3 μl of the Aβ solution was slowly injected, held for 5 to 10 seconds, and then the needle was gently withdrawn (Aβ dose: 409.8 μmol/3 μl/mouse).

As a result, the following three mouse groups were prepared:
a group of mice into which a control (physiological saline) administration sample had been orally administered beforehand, which was then subjected to ICV administration of Aβ to become ICV Aβ mice, and then subjected to oral administration of a control administration sample (hereafter referred to as “control-administered mice”);
a group of mice into which a clove extract administration sample had been orally administered beforehand, which was then subjected to ICV administration of Aβ to become ICV Aβ mice, and then subjected to oral administration of a clove extract administration sample (hereafter referred to as “clove-extract-administered mice”); and
a group of mice into which an allspice extract administration sample had been orally administered beforehand, which was then subjected to ICV administration of Aβ to become ICV Aβ mice, and then subjected to oral administration of an allspice extract administration sample (hereafter referred to as “allspice-extract-administered mice”).
   (viii) Evaluation of Cognitive Function in Mice Using Y-Maze Test

The cognitive function of the control-administered mice, the clove-extract-administered mice, and the allspice-extract-administered mice was evaluated on day 19 or 20 after their arrival using the Y-maze test, which can evaluate the spatial working memory of mice. The preventive effect of sample administration on cognitive function decline was evaluated.

In the Y-maze test, mice were allowed to explore a Y-maze for 5 minutes, and the total number of times entering each arm and the number of times entering three different arms consecutively were recorded. The entry criterion was that the nose had passed halfway through the arm, and the alternation behavior rate, which is an index of spatial working memory, was calculated using the following formula. Mice with high spatial working memory exhibit high alternation behavior rates.

Alternation behavior rate (%)=Number of times entering three different arms consecutively/Total number of entries−2×100 Formula (1)

The alternation behavior rate of each mouse is shown in Table 4. Mice into which the administration samples of extracts of the family Myrtaceae (allspice extract and clove extract) were orally administered before intracerebral administration of Aβ (allspice-extract-administered mice or clove-extract-administered mice) had higher alternation behavior rates than mice into which the control (physiological saline) administration sample was orally administered before intracerebral administration of Aβ (control-administered mice). This demonstrates that allspice extract and clove extract prevent/improve spatial working memory loss. From the above results, it was confirmed that extracts of the family Myrtaceae have the effects of preventing/improving the loss of spatial working memory and the effects of preventing cognitive function decline and improving cognitive function.

TABLE 4

| | Average alternation |
|---|---|
| Mouse (4 mice per group) | behavior rate (n = 4) |
| Control administration mouse | 31.8% |
| Allspice extract administration mouse | 53.5% |
| Clove extract administration mouse | 74.2% |

The agent for suppressing Aβ aggregation/deposition of one or more embodiments of the present invention can exhibit a favorable effect of suppressing Aβ aggregation/deposition in body tissues (for example, the surface and/or inside of brain tissue cells in a living organism).

Furthermore, the agent for suppressing Aβ aggregation/deposition of one or more embodiments of the present invention can exhibit an effect of suppressing a decline in/improving the cognitive function of a subject, which is the subject's function to recognize things via the brain, and thus can be used as an agent for suppressing cognitive function decline/improving cognitive function.

All publications, patents, and patent applications cited herein are incorporated herein by reference in their entirety.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

1. An agent for suppressing amyloid β aggregation and/or deposition, consisting of an extract of a plant belonging to the family Myrtaceae.

2. The agent for suppressing aggregation and/or deposition according to claim 1,
wherein the agent is identified as suppressing amyloid β aggregation and/or deposition in an evaluation system comprising:
a step of coexisting and culturing amyloid β with a label, a test substance, and a cerebral organoid; and
a step of quantifying amyloid β aggregated on a surface of and/or inside the cerebral organoid using the label as an indicator.

3. The agent for suppressing aggregation and/or deposition according to claim 2, having a suppression rate of 50% or more as compared with a control plot, to which the test substance is not added, in the evaluation system.

4. The agent for suppressing aggregation and/or deposition according to claim 1, wherein the plant belonging to the family Myrtaceae is guava, Eucalyptus robusta, clove, or allspice.

5. The agent for suppressing aggregation and/or deposition according to claim 4, wherein the extract is derived from a pulverized product subjected to solvent extraction, wherein the pulverized product is a pulverized clove.

6. The agent for suppressing aggregation and/or deposition according to claim 5, wherein the pulverized product consists of particles having a particle size of 5 mm or less.

7. The agent for suppressing aggregation and/or deposition according to claim 5, wherein the pulverized product consists of particles having a particle size of 0.5 mm or more.

8. The agent for suppressing aggregation and/or deposition according to claim 5, wherein the pulverized product consists of particles having a particle size range of 0.5 mm or more and 3 mm or less.

9. The agent for suppressing aggregation and/or deposition according to claim 1, wherein the agent suppresses the amyloid β aggregation and/or deposition on a surface of and/or inside brain tissue cell in a living organism.

10. A composition comprising an agent for suppressing cognitive function decline and/or improving cognitive function agent for suppressing cognitive function decline and/or improving cognitive function in a subject, the agent comprising extract of a plant belonging to the family Myrtaceae.

11. The composition according to claim 10, wherein the plant is clove or allspice.

12. A method for producing an agent for suppressing amyloid β aggregation and/or deposition, comprising:
a step of obtaining an extract by subjecting a plant belonging to the family Myrtaceae to solvent extraction.

13. The method for producing an agent for suppressing aggregation and/or deposition according to claim 12, wherein the plant belonging to the family Myrtaceae is guava, Eucalyptus robusta, clove, or allspice.

14. The method for producing an agent for suppressing aggregation and/or deposition according to claim 13, wherein the step of obtaining an extract is a step of obtaining the extract by subjecting a pulverized product, which is obtained by pulverizing clove, to solvent extraction.

15. The method for producing an agent for suppressing aggregation and/or deposition according to claim 14, wherein the pulverized product obtained by pulverizing clove, has a maximum particle size of 5 mm or less.

16. The method for producing an agent for suppressing aggregation and/or deposition according to claim 14, wherein the pulverized product obtained by pulverizing clove, has a maximum particle size of 0.5 mm or more.

17. The method for producing an agent for suppressing aggregation and/or deposition according to claim 14, wherein the pulverized product obtained by pulverizing clove, has a maximum particle size range of 0.5 mm or more and 3 mm or less.

18. The method for producing an agent for suppressing aggregation and/or deposition according to claim 12, wherein a solvent used in the solvent extraction is an organic solvent, water, or a mixture thereof.

19. The method for producing an agent for suppressing aggregation and/or deposition according to claim 12, wherein a solvent of the solvent extraction is an ethanol aqueous solution.

20. A method for producing an agent for suppressing cognitive function decline and/or improving cognitive function in a subject, comprising obtaining an extract by subjecting a plant belonging to the family Myrtaceae to solvent extraction.

21. The method for producing an agent for suppressing cognitive function decline and/or improving cognitive function in a subject according to claim 20, wherein the plant is clove or allspice.
